(19) United States
(12) Patent Application Publication
(10) Pub. No.: US 2002/0168363 A1
(43) Pub. Date: Nov. 14, 2002

(54) Integrin/adhesion antagonists

(75) Inventors: Ulrich Feige, Newbury Park, CA (US); Tadahiko Kohno, Thousand Oaks, CA (US); David Lee Lacey, Newbury Park, CA (US); Thomas Charles Boone, Newbury Park, CA (US)

Correspondence Address: AMGEN INCORPORATED, MAIL STOP 27-4-A, ONE AMGEN CENTER DRIVE, THOUSAND OAKS, CA, 91320-1799, US

(73) Assignee: Amgen Inc.

(21) Appl. No.: 09/840,277

(22) Filed: Apr. 23, 2001

Related U.S. Application Data
(60) Provisional application No. 60/201,394, filed on May 3, 2000.
(60) Provisional application No. 60/198,919, filed on Apr. 21, 2000.

(51) Int. Cl. A61K 39/395

(52) U.S. Cl. 424/146.1; 514/12

(57) ABSTRACT

The present invention concerns fusion of half-life extending vehicles, preferably Fc domains, with peptide sequences that act as antagonists of integrins, selectins, cell adhesion molecules, or their respective receptors. Linkage to the vehicle increases the half-life of the peptide, which otherwise would be quickly degraded in vivo. The peptide may be an existing peptide or a peptide selected by phage display, E. coli display, ribosome display, RNA-peptide screening, chemical-peptide screening, or other methods.

This application claims the benefit of U.S. Provisional Application No. 60/201,394, filed May 3, 2000, and U.S. Provisional Application No. 60/198,919, filed Apr. 21, 2000, which are hereby incorporated by reference.

### BACKGROUND OF THE INVENTION

A need exists for recombinant or modified therapeutic agents having anti-integrin activity.

Recombinant proteins are an emerging class of therapeutic agents. Such recombinant therapeutics have engendered advances in protein formulation and chemical modification. Such modifications can protect therapeutic proteins, primarily by blocking their exposure to proteolytic enzymes. Protein modifications may also increase the therapeutic protein's stability, circulation time, and biological activity. A review article describing protein modification and fusion proteins is Francis (1992), Focus on Growth Factors 3:4-10 (Mediscript, London), which is hereby incorporated by reference.

One useful modification is combination with the “Fc” domain of an antibody. Antibodies comprise two functionally independent parts, a variable domain known as “Fab”, which binds antigen, and a constant domain known as “Fc”, which links to such effector functions as complement activation and attack by phagocytic cells. An Fc has a long serum half-life, whereas an Fab is short-lived. Capon et al. (1989), Nature 337: 525-31. When constructed together with a therapeutic protein, an Fc domain can provide longer half-life or incorporate such functions as Fc receptor binding, protein A binding, complement fixation and perhaps even placental transfer. Id. Table 1 summarizes use of Fc fusions known in the art.

TABLE 1
Fc fusion with therapeutic proteins

| Form | Fusion | Therapeutic | |
|---|---|---|---|
| of Fc | partner | implications | Reference |
| IgG1 | N-terminus of | Hodgkin's disease; | U.S. Pat. No. |
| | CD30-L | anaplastic lymphoma; | 5,480,981 |
| | | T-cell leukemia | |
| Murine | IL-10 | anti-inflammatory; | Zheng et al. (1995), |
| Fcγ2a | | transplant rejection | J. Immunol. 154: |
| | | | 5590-600 |
| IgG1 | TNF receptor | septic shock | Fisher et al. (1996), |
| | | | N. Engl. J. Med, |
| | | | 334: 1697-1702; |
| | | | Van Zee, K. et al. |
| | | | (1996), J. Immunol, |
| | | | 156: 2221-30 |
| IgG, IgA, | TNF receptor | inflammation, | U.S. Pat. No. |
| IgM, or | | autoimmune disorders | 5,808,029, issued |
| IgE (ex- | | | September 15, 1998 |
| cluding | | | |
| the first | | | |
| domain) | | | |
| IgG1 | CD4 receptor | AIDS | Capon et al. (1989), |
| | | | Nature 337: 525-31 |
| IgG1, | N-terminus | anti-cancer, | Harvill et al. (1995), |
| IgG3 | of IL-2 | antiviral | Immunotech. 1: |
| | | | 95-105 |
| IgG1 | C-terminus | osteoarthritis; | WO 97/23614, |
| | of OPG | bone density | published |
| | | | July 3, 1997 |
| IgG1 | N-terminus | anti-obesity | PCT/US 97/23183, |
| | of leptin | | filed December 11, |
| | | | 1997 |
| Human Ig | CTLA-4 | autoimmune disorders | Linsley (1991), |
| Cγ1 | | | J. Exp. Med. 174: |
| | | | 561-9 |

A much different approach to development of therapeutic agents is peptide library screening. The interaction of a protein ligand with its receptor often takes place at a relatively large interface. However, as demonstrated for human growth hormone and its receptor, only a few key residues at the interface contribute to most of the binding energy. Clackson et al. (1995), Science 267: 383-6. The bulk of the protein ligand merely displays the binding epitopes in the right topology or serves functions unrelated to binding. Thus, molecules of only “peptide” length (2 to 40 amino acids) can bind to the receptor protein of a given large protein ligand. Such peptides may mimic the bioactivity of the large protein ligand (“peptide agonists”) or, through competitive binding, inhibit the bioactivity of the large protein ligand (“peptide antagonists”).

Phage display peptide libraries have emerged as a powerful method in identifying such peptide agonists and antagonists. See, for example, Scott et al. (1990), Science 249: 386; Devlin et al. (1990), Science 249: 404; U.S. Pat. No. 5,223,409, issued Jun. 29, 1993; U.S. Pat. No. 5,733,731, issued Mar. 31, 1998; U.S. Pat. No. 5,498,530, issued Mar. 12, 1996; U.S. Pat. No. 5,432,018, issued Jul. 11, 1995; U.S. Pat. No. 5,338,665, issued Aug. 16, 1994; U.S. Pat. No. 5,922,545, issued Jul. 13, 1999; WO 96/40987, published Dec. 19, 1996; and WO 98/15833, published Apr. 16, 1998 (each of which is incorporated by reference). In such libraries, random peptide sequences are displayed by fusion with coat proteins of filamentous phage. Typically, the displayed peptides are affinity-eluted against an antibody-immobilized extracellular domain of a receptor. The retained phages may be enriched by successive rounds of affinity purification and repropagation. The best binding peptides may be sequenced to identify key residues within one or more structurally related families of peptides. See, e.g., Cwirla et al. (1997), Science 276: 1696-9, in which two distinct families were identified. The peptide sequences may also suggest which residues may be safely replaced by alanine scanning or by mutagenesis at the DNA level. Mutagenesis libraries may be created and screened to further optimize the sequence of the best binders. Lowman (1997), Ann. Rev. Biophys. Biomol. Struct. 26: 401-24.

Other methods compete with phage display in peptide research. A peptide library can be fused to the carboxyl terminus of the lac repressor and expressed in E. coli. Another E. coli-based method allows display on the cell's outer membrane by fusion with a peptidoglycan-associated lipoprotein (PAL). Hereinafter, these and related methods are collectively referred to as “E. coli display.” Another biological approach to screening soluble peptide mixtures uses yeast for expression and secretion. See Smith et al. (1993), Mol. Pharmacol. 43: 741-8. Hereinafter, the method of Smith et al. and related methods are referred to as “yeast-based screening.” In another method, translation of random RNA is halted prior to ribosome release, resulting in a library of polypeptides with their associated RNA still attached. Hereinafter, this and related methods are collectively referred to as “ribosome display.” Other methods employ chemical linkage of peptides to RNA; see, for example, Roberts & Szostak (1997), Proc. Natl. Acad. Sci. USA, 94: 12297-303. Hereinafter, this and related methods are collectively referred to as “RNA-peptide screening.” Chemically derived peptide libraries have been developed in which peptides are immobilized on stable, non-biological materials, such as polyethylene rods or solvent-permeable resins. Another chemically derived peptide library uses photolithography to scan peptides immobilized on glass slides. Hereinafter, these and related methods are collectively referred to as “chemical-peptide screening.” Chemical-peptide screening may be advantageous in that it allows use of D-amino acids and other unnatural analogues, as well as non-peptide elements. Both biological and chemical methods are reviewed in Wells & Lowman (1992), Curr. Opin. Biotechnol. 3: 355-62.

In the case of known bioactive peptides, rational design of peptide ligands with favorable therapeutic properties can be completed. In such an approach, one makes stepwise changes to a peptide sequence and determines the effect of the substitution upon bioactivity or a predictive biophysical property of the peptide (e.g., solution structure). Hereinafter, these techniques are collectively referred to as “rational design.” In one such technique, one makes a series of peptides in which one replaces a single residue at a time with alanine. This technique is commonly referred to as an “alanine walk” or an “alanine scan.” When two residues (contiguous or spaced apart) are replaced, it is referred to as a “double alanine walk.” The resultant amino acid substitutions can be used alone or in combination to result in a new peptide entity with favorable therapeutic properties.

Structural analysis of protein-protein interaction may also be used to suggest peptides that mimic the binding activity of large protein ligands. In such an analysis, the crystal structure may suggest the identity and relative orientation of critical residues of the large protein ligand, from which a peptide may be designed. See, e.g., Takasaki et al. (1997), Nature Biotech. 15: 1266-70. Hereinafter, these and related methods are referred to as “protein structural analysis.” These analytical methods may also be used to investigate the interaction between a receptor protein and peptides selected by phage display, which may suggest further modification of the peptides to increase binding affinity.

Conceptually, one may discover peptide mimetics of any protein using phage display and the other methods mentioned above. These methods have been used for epitope mapping, for identification of critical amino acids in protein-protein interactions, and as leads for the discovery of new therapeutic agents. E.g., Cortese et al. (1996), Curr. Opin. Biotech. 7: 616-21. Peptide libraries have been used most often in immunological studies, such as epitope mapping. Kreeger (1996), The Scientist 10(13): 19-20.

Of particular interest here is use of peptide libraries and other techniques in the discovery of peptides that inhibit integrins, selecting, cellular adhesion molecules, or their respective receptors. A number of such peptides identified in the art are summarized in Table 2. For randomly generated peptides, peptide libraries typically were screened for binding to a receptor for an integrin ligand (e.g., α4β1). For purposes of this application, these molecules are collectively termed, “Integrin/adhesion antagonists.”

In Table 2, the protein listed in the left side column in this table may be bound by the associated peptide or mimicked by the associated peptide. The structure and activity of the peptides are described in the listed publications, each of which is hereby incorporated by reference in its entirety. The middle column describes the pharmacologic activity of the peptides, and in some instances is followed by a shorthand term in parentheses.

TABLE 2
Integrin/adhesion antagonist peptides

| Binding | | |
|---|---|---|
| partner/ | | |
| protein of | Pharmacologic | |
| interest | activity | Reference |
| laminin | integrin antagonist, | Saiki, I. et al. (1989), |
| | useful in treating | Br. J. Cancer 60: 722-8; |
| | tumor growth, tumor | Mu et al. (1999), BBRC 255; |
| | metastasis | 75-79; Nomizu et al. (1993), |
| | | Cancer Research 53: 3459-61. |
| vinculin | cell adhesion processes- | Adey et al. (1997), |
| | cell growth, differentiation, | Biochem. J. 324: 523-8 |
| | wound healing, tumor | |
| | metastasis (“vinculin | |
| | binding”) | |
| selectins | neutrophil adhesion; | Martens et al. (1995), |
| | inflammatory diseases | J. Biol. Chem. 270: 21129-36; |
| | (“selectin antagonist”) | European patent application |
| | | EP 0 714 912, published |
| | | June 5, 1996 |
| integrins | tumor-homing; treatment | International applications |
| | for conditions related to | WO 95/14714, published |
| | integrin-mediated cellular | June 1, 1995; WO 97/08203, |
| | events, including platelet | published March 6, 1997; |
| | aggregation, thrombosis, | WO 98/10795, published |
| | wound healing, osteoporosis, | March 19, 1998; |
| | tissue repair, angiogenesis | WO 99/24462, published |
| | (e.g., for treatment of | May 20, 1999; Kraft et al. |
| | cancer), and tumor invasion | (1999), J. Biol. Chem. 274: |
| | (“integrin-binding”) | 1979-1985 Gan (1988), |
| Echistatin | inhibition of platelet | J. Biol. Chem., 263: |
| | aggregation | 19827-32. |

Peptides identified by peptide library screening have been regarded as “leads” in development of therapeutic agents rather than as therapeutic agents themselves. Like other proteins and peptides, they would be rapidly removed in vivo either by renal filtration, cellular clearance mechanisms in the reticuloendothelial system, or proteolytic degradation. Francis (1992), Focus on Growth Factors 3: 4-11. As a result, the art presently uses the identified peptides to validate drug targets or as scaffolds for design of organic compounds that might not have been as easily or as quickly identified through chemical library screening. Lowman (1997), Ann. Rev. Biophys. Biomol. Struct. 26: 401-24; Kay et al. (1998), Drug Disc. Today 3: 370-8.

### SUMMARY OF THE INVENTION

The present invention concerns therapeutic agents that have integrin antagonist activity, including activity of known peptides but with better pharmaceutical characteristics (e.g., half-life). In accordance with the present invention, such compounds comprise:

a. an integrin/adhesion antagonist peptide; and

b. a vehicle, such as a polymer (e.g., PEG or dextran) or an Fc domain, which is preferred;

wherein the vehicle is covalently attached to the integrin/adhesion antagonist. The vehicle and the integrin/adhesion antagonist may be linked through the N- or C-terminus of the integrin/adhesion antagonist, as described further below. The preferred vehicle is an Fc domain, and the preferred Fc domain is an IgG Fc domain. Integrin/adhesion antagonists can be generated by phage display, RNA-peptide screening and the other techniques mentioned herein.

The present invention also concerns a process by which the in vivo half-life of one or more biologically active peptides is increased by fusion with a vehicle. In this invention, pharmacologically active compounds are prepared by a process comprising:

a. selecting at least one integrin/adhesion antagonist peptide; and

b. preparing a pharmacologic agent comprising at least one vehicle covalently linked to at least one amino acid sequence of the selected peptide.

The preferred vehicle is an Fc domain. The peptides screened in step (a) are preferably expressed in a phage display library. The vehicle and the peptide may be linked through the N- or C-terminus of the peptide or the vehicle, as described further below. Preferred antagonist domains comprise the amino acid sequences described hereinafter in SEQ ID NOS: 7 to 21 and in Tables 3, 4, and 5. Additional antagonist domains can be generated by such techniques as rational design, yeast-based screening, rational design, protein structural analysis, phage display, and RNA-peptide screening. Derivatives of the above compounds (described below) are also encompassed by this invention.

The compounds of this invention may be prepared by standard synthetic methods, recombinant DNA techniques, or any other methods of preparing peptides and fusion proteins. Compounds of this invention that encompass non-peptide portions may be synthesized by standard organic chemistry reactions, in addition to standard peptide chemistry reactions when applicable.

The primary use contemplated is as therapeutic or prophylactic agents. The vehicle-linked peptides may have activity comparable to or even greater than natural ligands or known peptides. In addition, natural ligand-based therapeutic agents might induce antibodies against the patient's own endogenous ligand; the vehicle-linked peptides avoid this pitfall by having little or typically no sequence identity with the natural ligand.

The compounds of this invention may be used for therapeutic or prophylactic purposes by formulating them with appropriate pharmaceutical carrier materials and administering an effective amount to a patient, such as a human (or other mammal) in need thereof. Other related aspects are also included in the instant invention.

Numerous additional aspects and advantages of the present invention will become apparent upon consideration of the figures and detailed description of the invention.

### BRIEF DESCRIPTION OF THE FIGURES

FIG. 1 shows exemplary Fc dimers that may be derived from an IgG1 antibody. “Fc” in the figure represents any of the Fc variants within the meaning of “Fc domain” herein. “X¹” and “X²” represent peptides or linker-peptide combinations as defined hereinafter. The specific dimers are as follows:

A, D: Single disulfide-bonded dimers. IgG1 antibodies typically have two disulfide bonds at the hinge region between the constant and variable domains. The Fc domain in FIGS. 2A and 2D may be formed by truncation between the two disulfide bond sites or by substitution of a cysteinyl residue with an unreactive residue (e.g., alanyl). In FIG. 2A, the Fc domain is linked at the amino terminus of the peptides; in 2D, at the carboxyl terminus.

B, E: Doubly disulfide-bonded dimers. This Fc domain may be formed by truncation of the parent antibody to retain both cysteinyl residues in the Fc domain chains or by expression from a construct including a sequence encoding such an Fc domain. In FIG. 2B, the Fc domain is linked at the amino terminus of the peptides; in 2E, at the carboxyl terminus.

C, F: Noncovalent dimers. This Fc domain may be formed by elimination of the cysteinyl residues by either truncation or substitution. One may desire to eliminate the cysteinyl residues to avoid impurities formed by reaction of the cysteinyl residue with cysteinyl residues of other proteins present in the host cell. The noncovalent bonding of the Fc domains is sufficient to hold together the dimer. Other dimers may be formed by using Fc domains derived from different types of antibodies (e.g., IgG2, IgM).

FIG. 2 shows the structure of preferred compounds of the invention that feature tandem repeats of pharmacologically active peptides. FIG. 2A shows a single chain molecule and may also represent the DNA construct for the molecule. FIG. 2B shows a dimer in which the linker-peptide portion is present on only one chain of the dimer. FIG. 2C shows a dimer having the peptide portion on both chains. The dimer of FIG. 2C will form spontaneously in certain host cells upon expression of a DNA construct encoding the single chain shown in FIG. 2A. In other host cells, the cells could be placed in conditions favoring formation of dimers or the dimers can be formed in vitro.

FIG. 3 shows exemplary nucleic acid and amino acid sequences (SEQ ID NOS: 1 and 2, respectively) of human IgG1 Fc that may be used in this invention.

FIGS. 4A and 4B show that Echistatin-Fc binds with high affinity to human αvβ3 in the solid phase binding assay. This assay is further described in Example 1 hereinafter.

FIGS. 5A and 5B show inhibition of ruthenium-labeled human fibrinogen (fibrinogen-ru) binding to GPIIb/IIIa with Echistatin-Fc. These experiments are further described in Example 1 hereinafter.

### DETAILED DESCRIPTION OF THE INVENTION

Definition of Terms

The terms used throughout this specification are defined as follows, unless otherwise limited in specific instances.

The term “comprising” means that a compound may include additional amino acids on either or both of the N- or C-termini of the given sequence. Of course, these additional amino acids should not significantly interfere with the activity of the compound.

The term “vehicle” refers to a molecule that prevents degradation and/or increases half-life, reduces toxicity, reduces immunogenicity, or increases biological activity of a therapeutic protein. Exemplary vehicles include an Fc domain (which is preferred) as well as a linear polymer (e.g., polyethylene glycol (PEG), polylysine, dextran, etc.); a branched-chain polymer (see, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published Oct. 28, 1993); a lipid; a cholesterol group (such as a steroid); a carbohydrate or oligosaccharide; or any natural or synthetic protein, polypeptide or peptide that binds to a salvage receptor. Vehicles are further described hereinafter.

The term “native Fc” refers to molecule or sequence comprising the sequence of a non-antigen-binding fragment resulting from digestion of whole antibody, whether in monomeric or multimeric form. The original immunoglobulin source of the native Fc is preferably of human origin and may be any of the immunoglobulins, although IgG1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. International applications WO 97/34631 (published Sep. 25, 1997) and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by other means.

The term “multimer” as applied to Fc domains or molecules comprising Fc domains refers to molecules having two or more polypeptide chains associated covalently, noncovalently, or by both covalent and non-covalent interactions. IgG molecules typically form dimers; IgM, pentamers; IgD, dimers; and IgA, monomers, dimers, trimers, or tetramers. Multimers may be formed by exploiting the sequence and resulting activity of the native Ig source of the Fc or by derivatizing (as defined below) such a native Fc.

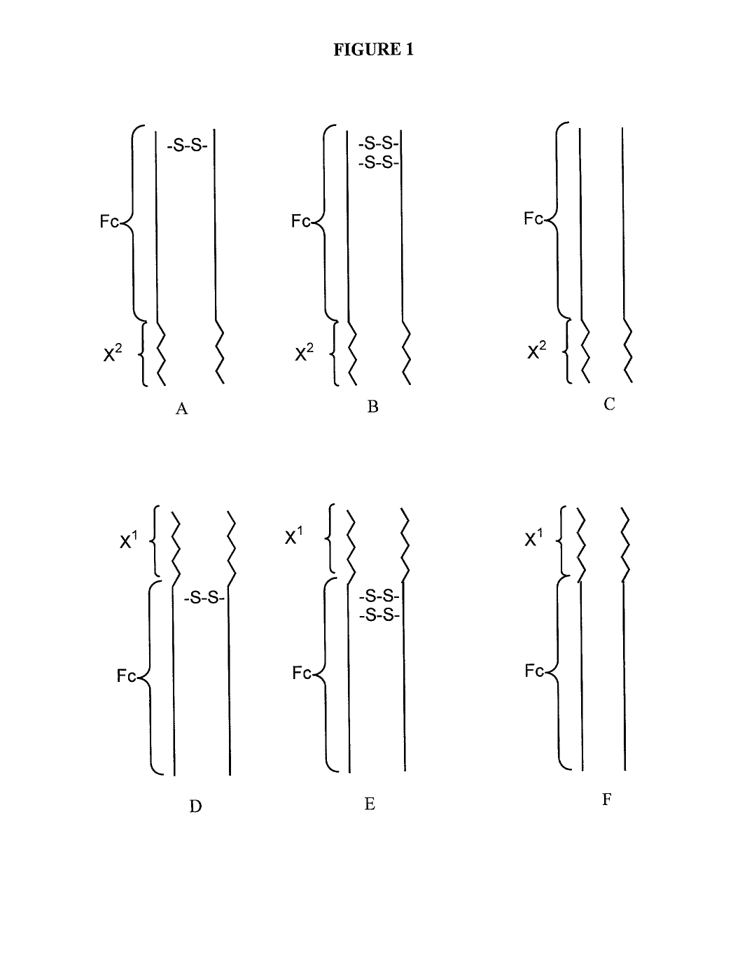

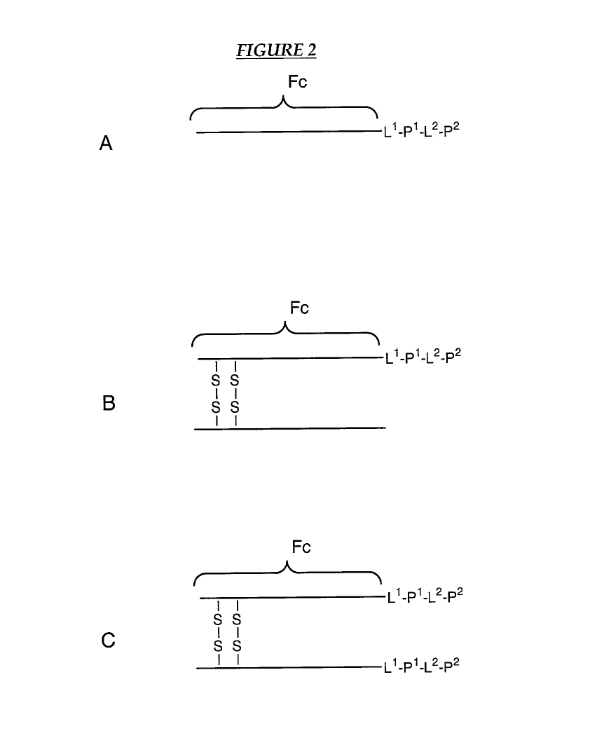

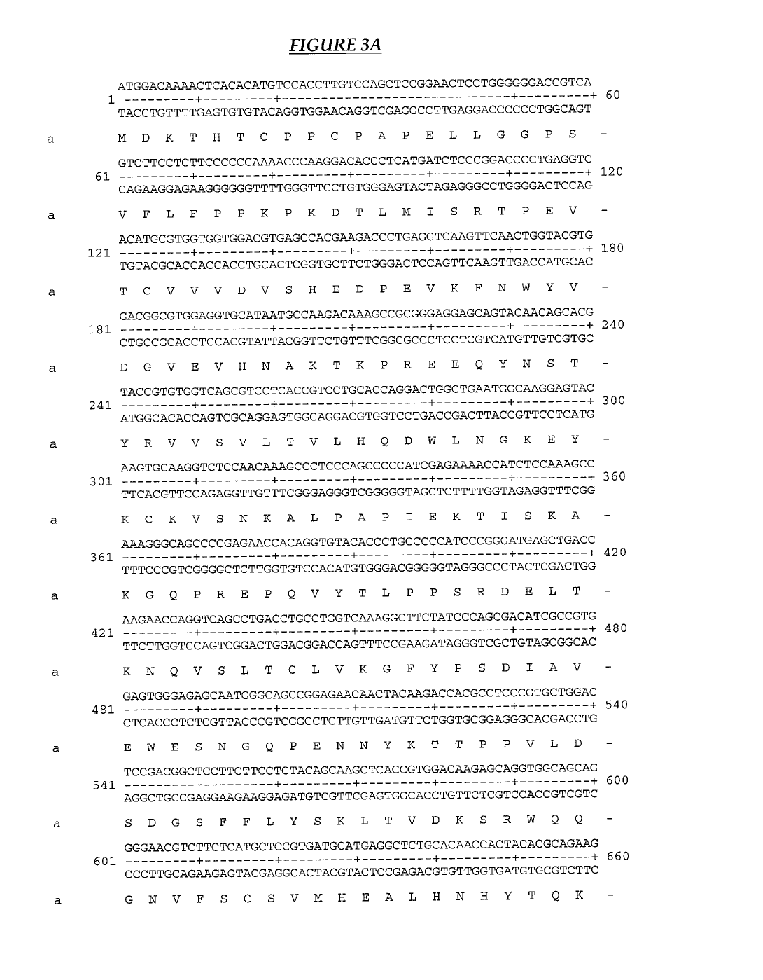

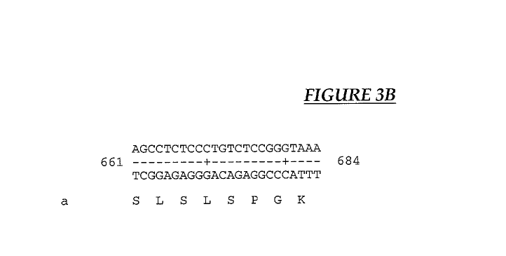

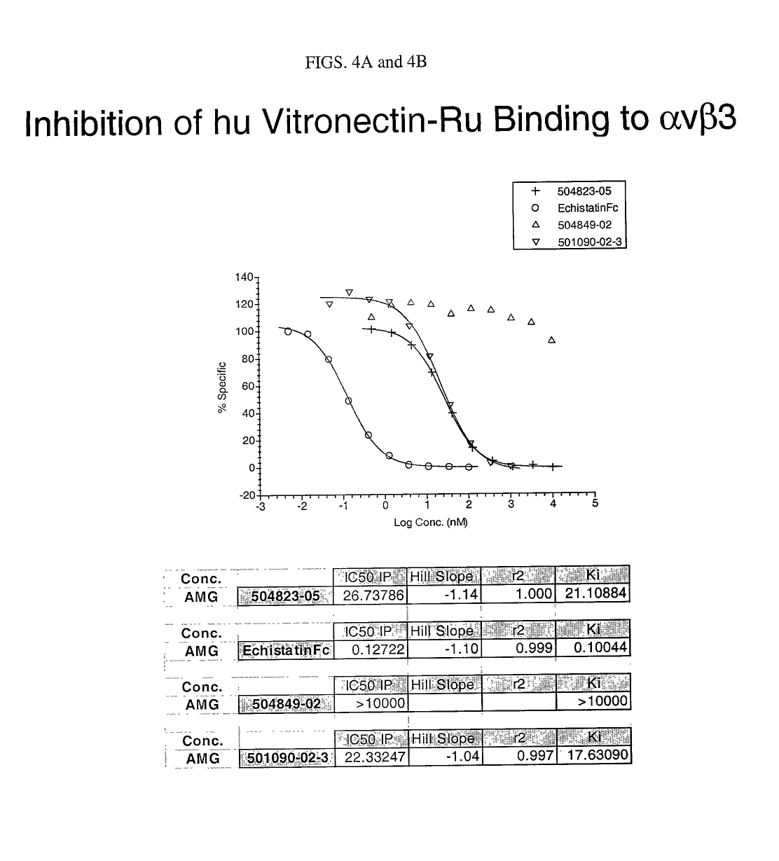

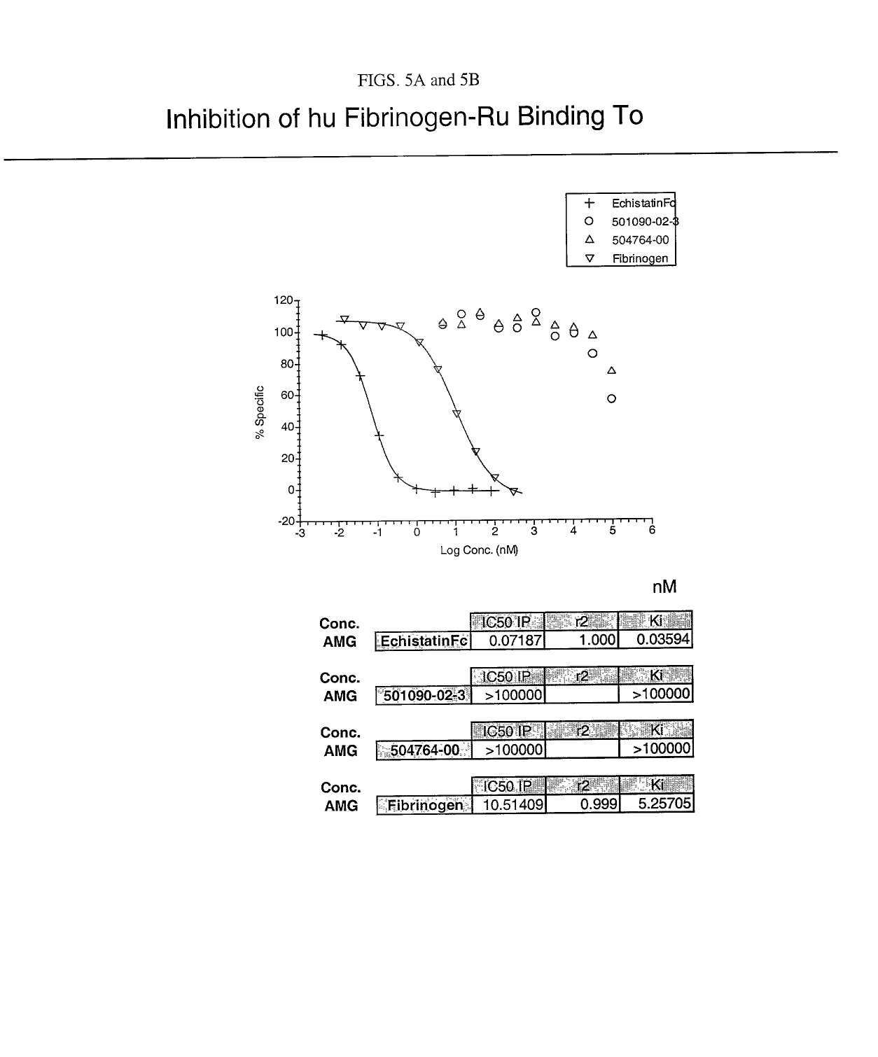

The term “dimer” as applied to Fc domains or molecules comprising Fc domains refers to molecules having two polypeptide chains associated covalently or non-covalently. Thus, exemplary dimers within the scope of this invention are as shown in FIG. 1.

The terms “derivatizing” and “derivative” or “derivatized” comprise processes and resulting compounds respectively in which (1) the compound has a cyclic portion; for example, cross-linking between cysteinyl residues within the compound; (2) the compound is cross-linked or has a cross-linking site; for example, the compound has a cysteinyl residue and thus forms cross-linked dimers in culture or in vivo; (3) one or more peptidyl linkage is replaced by a non-peptidyl linkage; (4) the N-terminus is replaced by —NRR¹, NRC(O)R¹, —NRC(O)OR¹, —NRS(O)₂R¹, —NHC(O)NHR, a succinimide group, or substituted or unsubstituted benzyloxycarbonyl-NH—, wherein R and R¹ and the ring substituents are as defined hereinafter; (5) the C-terminus is replaced by —C(O)R² or —NR³R⁴ wherein R², R³ and R⁴ are as defined hereinafter; and (6) compounds in which individual amino acid moieties are modified through treatment with agents capable of reacting with selected side chains or terminal residues. Derivatives are further described hereinafter.

The term “peptide” refers to molecules of 2 to 60 amino acids, with molecules of 3 to 20 amino acids preferred and those of 6 to 15 amino acids most preferred. Exemplary peptides may be randomly generated by any of the methods cited above, carried in a peptide library (e.g., a phage display library), or derived by digestion of proteins.

The term “randomized” as used to refer to peptide sequences refers to fully random sequences (e.g., selected by phage display methods) and sequences in which one or more residues of a naturally occurring molecule is replaced by an amino acid residue not appearing in that position in the naturally occurring molecule. Exemplary methods for identifying peptide sequences include phage display, E. coli display, yeast-based screening, ribosome display, RNA-peptide screening, chemical screening, rational design, protein structural analysis, and the like.

The term “pharmacologically active” means that a substance so described is determined to have activity that affects a medical parameter (e.g., blood pressure, blood cell count, cholesterol level) or disease state (e.g., cancer, autoimmune disorders). Thus, pharmacologically active peptides comprise agonistic or mimetic and antagonistic peptides as defined below.

The term “-antagonist peptide” or “inhibitor peptide” refers to a peptide that blocks or in some way interferes with the biological activity of the associated protein of interest, or has biological activity comparable to a known antagonist or inhibitor of the associated protein of interest.

The term “integrin/adhesion antagonist” comprises peptides that inhibit or down-regulate the activity of integrins, selecting, cell adhesion molecules, integrin receptors, selectin receptors, or cell adhesion molecule receptors. Exemplary integrin/adhesion antagonists comprise laminin, echistatin, the peptides described in SEQ ID NOS: 7 to 21 hereinafter, the peptides in Tables 3, 4, and 5 hereinafter, and those described in the references in Table 2. Those of ordinary skill in the art appreciate that each of these references enables one to select different peptides than actually disclosed herein by following the disclosed procedures with different peptide libraries.

Additionally, physiologically acceptable salts of the compounds of this invention are also encompassed herein. By “physiologically acceptable salts” is meant any salts that are known or later discovered to be pharmaceutically acceptable. Some specific examples are: acetate; trifluoroacetate; hydrohalides, such as hydrochloride and hydrobromide; sulfate; citrate; tartrate; glycolate; and oxalate.

Structure of compounds

In General. In the compositions of matter prepared in accordance with this invention, the peptide may be attached to the vehicle through the peptide's N-terminus or C-terminus. Thus, the vehicle-peptide molecules of this invention may be described by the following formula I:

(X¹)ₐ—F¹—(X²)_{b} I

wherein:

F¹ is a vehicle (preferably an Fc domain);

X¹ and X² are each independently selected from —(L¹)_{c}—P¹, —(L¹)_{c}—P¹—(L²)_{d}—P², —(L¹)_{c}—P¹—(L²)_{d}—P²—(L³)ₑ—P³, and —(L¹)_{c}—P¹—(L²)_{d}—P²—(L³)ₑ—P³—(L⁴)_{f}—P⁴

P¹, P², P³, and P⁴ are each independently sequences of integrin/adhesion antagonist peptides;

L¹, L², L³, and L⁴ are each independently linkers; and

a, b, c, d, e, and f are each independently 0 or 1, provided that at least one of a and b is 1.

Thus, compound I comprises preferred compounds of the formula II

X¹—F¹

and multimers thereof wherein F¹ is an Fc domain and is attached at the C-terminus of X¹,

F¹—X² III

and multimers thereof wherein F¹ is an Fc domain and is attached at the N-terminus of X²;

F¹—(L¹)_{c}—P¹ IV

and multimers thereof wherein F¹ is an Fc domain and is attached at the N-terminus of —(L¹)_{c}—P¹; and

F¹—(L¹)_{c}—P¹—(L²)_{d}—P² V

and multimers thereof wherein F¹ is an Fc domain and is attached at the N-terminus of —L¹—P¹—L²—P².

Peptides. Any number of integrin/adhesion antagonist peptides may be used in conjunction with the present invention. Targeting peptides are also of interest, including tumor-homing peptides, cell-type specific peptides and the like. All of these classes of peptides may be discovered by methods described in the references cited in this specification and other references.

Phage display, in particular, is useful in generating peptides for use in the present invention. It has been stated that affinity selection from libraries of random peptides can be used to identify peptide ligands for any site of any gene product. Dedman et al. (1993), J. Biol. Chem. 268: 23025-30. Phage display is particularly well suited for identifying peptides that bind to such proteins of interest as cell surface receptors or any proteins having linear epitopes. Wilson et al. (1998), Can. J. Microbiol. 44: 313-29; Kay et al. (1998), Drug Disc. Today 3: 370-8. Such proteins are extensively reviewed in Herz et al. (1997), J. Receptor & Signal Transduction Res. 17(5): 671-776, which is hereby incorporated by reference. Such proteins of interest are preferred for use in this invention.

Particular proteins of interest as targets for peptide generation in the present invention are integrins, adhesion molecules, and receptors for integrins or adhesion molecules (e.g., αvβ3, αVβ1).

Peptides particularly of interest for use in the present invention include laminin, which has the sequence

| | |
|---|---|
| YIGSR | (SEQ ID NO:7) |

echistatin, which has the sequence

| | |
|---|---|
| ECESGPCCRNCKFLKEGTICKRARGDDMDDYCNGKTCDCPRNPHKGPAT | (SEQ ID NO:8) |

RGD, NGR and derivatives thereof having the sequences

| | |
|---|---|
| RX₁ETX₂WX₃ | (SEQ ID NO:9) |
| RX₁ETX₂WX₃ | (SEQ ID NO:10) |
| CX₁X₂RLDX₃X₄C | (SEQ ID NO:11) |
| CXXRGDC | (SEQ ID NO:12) |
| X₁X₂X₃RGDX₄X₅X₆ | (SEQ ID NO:13) |
| CX₂CRGDCX₅C | (SEQ ID NO:14) |
| X₁X₂DDX₄X₅X₇X₈ | (SEQ ID NO:15) |
| X₁X₂X₃DDX₄X₅X₆X₇X₈ | (SEQ ID NO:16) |

in which the substituents X₁, X₂, X₃, X₄, X₅, X₆, X₇, and X₈ are as defined in International applications WO 95/14714, published Jun. 1, 1995 and WO 97/08203, published Mar. 6, 1997, which are incorporated by reference in their entirety.

Also of particular interest for use in this invention are vinculin binding peptides and selectin antagonist peptides of the formulae

| | |
|---|---|
| RKXNXXWTWVGTXKXLTEE | (SEQ ID NO:17) |
| CXXXYTXLVAIQNKXE | (SEQ ID NO:18) |
| RKXXXXWXWVGTXKXLTXE | (SEQ ID NO:19) |
| AXNWXXXEPNNXXXED | (SEQ ID NO:20) |
| XKXKTXEAXNWXX | (SEQ ID NO:21) |

in which “X” refers to any naturally occurring amino acid residue.

Exemplary peptides for this invention appear in Tables 3, 4, 5 and 6 below. These peptides may be prepared by methods disclosed in the art. Single letter amino acid abbreviations are used. The X in these sequences (and throughout this specification, unless specified otherwise in a particular instance) means that any of the 20 naturally occurring amino acid residues may be present. Any of these peptides may be linked in tandem (i.e., sequentially), with or without linkers, with peptides of the same sequence or different sequences. Any peptide containing a cysteinyl residue may be cross-linked with another Cys-containing peptide, either or both of which may be linked to a vehicle. A few cross-linked examples are provided in the table. Any peptide having more than one Cys residue may form an intrapeptide disulfide bond, as well. In the “SEQ ID NO.” column, “NR” means that no sequence listing is required for the given sequence.

TABLE 3
Integrin-antagonist peptide sequences

| | SEQ.ID |
|---|---|
| Sequence/structure | NO: |
| CLCRGDCIC | 22 |
| CWDDGWLC | 23 |
| CWDDLWWLC | 24 |
| CWDDGLMC | 25 |
| CWDDGWMC | 26 |
| CSWDDGWLC | 27 |
| CPDDLWWLC | 28 |
| NGR | 29 |
| GSL | 30 |
| RGD | 31 |
| CGRECPRLCQSSC | 32 |
| GNGRCVSGCAGRC | 33 |
| CLSGSLSC | 34 |
| GSL | 35 |
| NGRAHA | 36 |
| CNGRC | 37 |
| CDCRGDCFC | 38 |
| CGSLVRC | 39 |
| DLXXL | 40 |
| RTDLDSLRTYTL | 41 |
| RTDLDSLRTY | 42 |
| RTDLDSLRT | 43 |
| RTDLDSLR | 44 |
| GDLDLLKLRLTL | 45 |
| GDLHSLRQLLSR | 46 |
| RDDLHMLRLQLW | 47 |
| SSDLHALKKRYG | 48 |
| RGDLKQLSELTW | 49 |
| CXXRGDC | 50 |
| STGGFDDVYDWARGVSSALTTTLVATR | 51 |
| STGGFDDVYDWARRVSSALTTTLVATR | 52 |
| SRGVNFSEWLYDMSAAMKEASNVFPSRRSR | 53 |
| SSQNWDMEAGVEDLTAAMLGLLSTIHSSSR | 54 |
| SSPSLYTQFLVNYESAATRIQDLLIASRPSR | 55 |
| SSTGWVDLLGALQRAADATRTSIPPSLQNSR | 56 |
| DVYTKKELIECARRVSEK | 57 |
| RGDGX | 58 |
| CRGDGXC | 59 |
| CARRLDAPC | 60 |
| CPSRLDSPC | 61 |
| CDCRGDCFC | 62 |
| CDCRGDCLC | 63 |
| RGDLAALSAPPV | 64 |

TABLE 4
Selectin antagonist peptide sequences

| | SEQ |
|---|---|
| Sequence/structure | ID NO: |
| DITWDQLWDLMK | 65 |
| DITWDELWKIMN | 66 |
| DYTWFELWDMMQ | 67 |
| QITWAQLWNMMK | 68 |
| DMTWHDLWTLMS | 69 |
| DYSWHDLWEMMS | 70 |
| EITWDQLWEVMN | 71 |
| HVSWEQLWDIMN | 72 |
| HITWDQLWRIMT | 73 |
| RNMSWLELWEHMK | 74 |
| AEWTWDQLWHVMNPAESQ | 75 |
| HRAEWLALWEQMSP | 76 |
| KKEDWLALWRIMSV | 77 |
| ITWDQLWDLMK | 78 |
| DITWDQLWDLMK | 79 |
| DITWDQLWDLMK | 80 |
| DITWDQLWDLMK | 81 |
| CQNRYTDLVAIQNKNE | 82 |
| AENWADNEPNNKRNNED | 83 |
| RKNNKTWTWVGTKKALTNE | 84 |
| KKALTNEAENWAD | 85 |
| CQXRYTDLVAIQNKXE | 86 |
| AENWADGEPNNKXNXED | 87 |

TABLE 5
Vinculin binding peptides

| | SEQ |
|---|---|
| Sequence/Structure | ID NO: |
| SSQNWDMEAGVEDLTAAMLGLLSTIHSSSR | 88 |
| SSPSLYTQFLVNYESAATRIQDLLIASRPSR | 89 |
| SSTGWVDLLGALQRAADATRTSIPPSLQNSR | 90 |
| DVYTKKELIECARRVSEK | 91 |
| STGGFDDVYDWARGVSSALTTTLVATR | 92 |
| STGGFDDVYDWARRVSSALTTTLVATR | 93 |
| SRGVNFSEWLYDMSAAMKEASNVFPSRRSR | 94 |

TABLE 6
Laminin-related peptide sequences

| | SEQ |
|---|---|
| Sequence/structure | ID NO: |
| YIGSRYIGSR [i.e., (YIGSR)₂] | 128 |
| YIGSRYIGSRYIGSR [i.e., (YIGSR)₃] | 129 |
| YIGSRYJGSRYIGSRYIGSR [i.e., (YIGSR)₄9 | 130 |
| YIGSRYIGSRYIGSRYIGSRYIGSR [i.e., (YIGSR)₅9 | 131 |
| IPCNNKGAHSVGLMWWMLAR | 132 |
| YIGSRREDVEILDVPDSGR | 133 |
| RGDRGDYIGSRRGD | 134 |
| YIGSRYIGSRYIGSRYIGSRYIGSR | 135 |
| REDVEILDVYIGSRPDSGR | 136 |
| YIGSRREDVEILDVPDSGR | 137 |

Vehicles. This invention requires the presence of at least one vehicle (F¹, F²) attached to a peptide through the N-terminus, C-terminus or a sidechain of one of the amino acid residues. Multiple vehicles may also be used; e.g., Fc's at each terminus or an Fc at a terminus and a PEG group at the other terminus or a sidechain.

An Fc domain is the preferred vehicle. The Fc domain may be fused to the N or C termini of the peptides or at both the N and C termini. For the TPO-mimetic peptides, molecules having the Fc domain fused to the N terminus of the peptide portion of the molecule are more bioactive than other such fusions, so fusion to the N terminus is preferred.

As noted above, Fc variants are suitable vehicles within the scope of this invention. A native Fc may be extensively modified to form an Fc variant in accordance with this invention, provided binding to the salvage receptor is maintained; see, for example WO 97/34631 and WO 96/32478. In such Fc variants, one may remove one or more sites of a native Fc that provide structural features or functional activity not required by the fusion molecules of this invention. One may remove these sites by, for example, substituting or deleting residues, inserting residues into the site, or truncating portions containing the site. The inserted or substituted residues may also be altered amino acids, such as peptidomimetics or D-amino acids. Fc variants may be desirable for a number of reasons, several of which are described below. Exemplary Fc variants include molecules and sequences in which:

1. Sites involved in disulfide bond formation are removed. Such removal may avoid reaction with other cysteine-containing proteins present in the host cell used to produce the molecules of the invention. For this purpose, the cysteine-containing segment at the N-terminus may be truncated or cysteine residues may be deleted or substituted with other amino acids (e.g., alanyl, seryl). In particular, one may truncate the N-terminal 20-amino acid segment of SEQ ID NO: 2 or delete or substitute the cysteine residues at positions 7 and 10 of SEQ ID NO: 2. Even when cysteine residues are removed, the single chain Fc domains can still form a dimeric Fc domain that is held together non-covalently.

2. A native Fc is modified to make it more compatible with a selected host cell. For example, one may remove the PA sequence near the N-terminus of a typical native Fc, which may be recognized by a digestive enzyme in E. coli such as proline iminopeptidase. One may also add an N-terminal methionine residue, especially when the molecule is expressed recombinantly in a bacterial cell such as E. coli. The Fc domain of SEQ ID NO: 2 (FIG. 3) is one such Fc variant.

3. A portion of the N-terminus of a native Fc is removed to prevent N-terminal heterogeneity when expressed in a selected host cell. For this purpose, one may delete any of the first 20 amino acid residues at the N-terminus, particularly those at positions 1, 2, 3, 4 and 5.

4. One or more glycosylation sites are removed. Residues that are typically glycosylated (e.g., asparagine) may confer cytolytic response. Such residues may be deleted or substituted with unglycosylated residues (e.g., alanine).

5. Sites involved in interaction with complement, such as the C1q binding site, are removed. For example, one may delete or substitute the EKK sequence of human IgG1. Complement recruitment may not be advantageous for the molecules of this invention and so may be avoided with such an Fc variant.

6. Sites are removed that affect binding to Fc receptors other than a salvage receptor. A native Fc may have sites for interaction with certain white blood cells that are not required for the fusion molecules of the present invention and so may be removed.

7. The ADCC site is removed. ADCC sites are known in the art; see, for example, Molec. Immunol. 29 (5): 633-9 (1992) with regard to ADCC sites in IgG1. These sites, as well, are not required for the fusion molecules of the present invention and so may be removed.

8. When the native Fc is derived from a non-human antibody, the native Fc may be humanized. Typically, to humanize a native Fc, one will substitute selected residues in the non-human native Fc with residues that are normally found in human native Fc. Techniques for antibody humanization are well known in the art.

Preferred Fc variants include the following. In SEQ ID NO: 2 (FIG. 3) the leucine at position 15 may be substituted with glutamate; the glutamate at position 99, with alanine; and the lysines at positions 101 and 103, with alanines. In addition, one or more tyrosine residues can be replaced by phenyalanine residues.

An alternative vehicle would be a protein, polypeptide, peptide, antibody, antibody fragment, or small molecule (e.g., a peptidomimetic compound) capable of binding to a salvage receptor. For example, one could use as a vehicle a polypeptide as described in U.S. Pat. No. 5,739,277, issued Apr. 14, 1998 to Presta et al. Peptides could also be selected by phage display for binding to the FcRn salvage receptor. Such salvage receptor-binding compounds are also included within the meaning of “vehicle” and are within the scope of this invention. Such vehicles should be selected for increased half-life (e.g., by avoiding sequences recognized by proteases) and decreased immunogenicity (e.g., by favoring non-immunogenic sequences, as discovered in antibody humanization).

As noted above, polymer vehicles may also be used for F¹ and F². Various means for attaching chemical moieties useful as vehicles are currently available, see, e.g., Patent Cooperation Treaty (“PCT”) International Publication No. WO 96/11953, entitled “N-Terminally Chemically Modified Protein Compositions and Methods,” herein incorporated by reference in its entirety. This PCT publication discloses, among other things, the selective attachment of water soluble polymers to the N-terminus of proteins.

A preferred polymer vehicle is polyethylene glycol (PEG). The PEG group may be of any convenient molecular weight and may be linear or branched. The average molecular weight of the PEG will preferably range from about 2 kiloDalton (“kD”) to about 100 kDa, more preferably from about 5 kDa to about 50 kDa, most preferably from about 5 kDa to about 10 kDa. The PEG groups will generally be attached to the compounds of the invention via acylation or reductive alkylation through a reactive group on the PEG moiety (e.g., an aldehyde, amino, thiol, or ester group) to a reactive group on the inventive compound (e.g., an aldehyde, amino, or ester group).

A useful strategy for the PEGylation of synthetic peptides consists of combining, through forming a conjugate linkage in solution, a peptide and a PEG moiety, each bearing a special functionality that is mutually reactive toward the other. The peptides can be easily prepared with conventional solid phase synthesis (see, for example, FIGS. 5 and 6 and the accompanying text herein). The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of water soluble polymer which may be used for protein modification. Dextrans are polysaccharide polymers comprised of individual subunits of glucose predominantly linked by α1-6 linkages. The dextran itself is available in many molecular weight ranges, and is readily available in molecular weights from about 1 kD to about 70 kD. Dextran is a suitable water soluble polymer for use in the present invention as a vehicle by itself or in combination with another vehicle (e.g., Fc). See, for example, WO 96/11953 and WO 96/05309. The use of dextran conjugated to therapeutic or diagnostic immunoglobulins has been reported; see, for example, European Patent Publication No. 0 315 456, which is hereby incorporated by reference. Dextran of about 1 kD to about 20 kD is preferred when dextran is used as a vehicle in accordance with the present invention.

Linkers. Any “linker” group is optional. When present, its chemical structure is not critical, since it serves primarily as a spacer. The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines. Other specific examples of linkers are:

(Gly)₃Lys(Gly)₄ (SEQ ID NO: 3);

(Gly)₃AsnGlySer(Gly)₂ (SEQ ID NO: 4);

(Gly)₃Cys(Gly)₄ (SEQ ID NO: 5); and

GlyProAsnGlyGly (SEQ ID NO: 6).

To explain the above nomenclature, for example, (Gly)₃Lys(Gly)₄ means Gly-Gly-Gly-Lys-Gly-Gly-Gly-Gly. Combinations of Gly and Ala are also preferred. The linkers shown here are exemplary; linkers within the scope of this invention may be much longer and may include other residues.

Non-peptide linkers are also possible. For example, alkyl linkers such as —NH—(CH₂)ₛ—C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker,

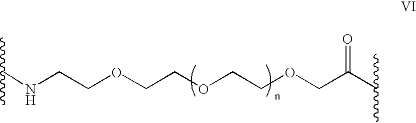

wherein n is such that the linker has a molecular weight of 100 to 5000 kD, preferably 100 to 500 kD. The peptide linkers may be altered to form derivatives in the same manner as described above.

Derivatives. The inventors also contemplate derivatizing the peptide and/or vehicle portion of the compounds. Such derivatives may improve the solubility, absorption, biological half life, and the like of the compounds. The moieties may alternatively eliminate or attenuate any undesirable side-effect of the compounds and the like. Exemplary derivatives include compounds in which:

1. The compound or some portion thereof is cyclic. For example, the peptide portion may be modified to contain two or more Cys residues (e.g., in the linker), which could cyclize by disulfide bond formation. For citations to references on preparation of cyclized derivatives, see Table 2.

2. The compound is cross-linked or is rendered capable of cross-linking between molecules. For example, the peptide portion may be modified to contain one Cys residue and thereby be able to form an intermolecular disulfide bond with a like molecule. The compound may also be cross-linked through its C-terminus, as in the molecule shown below.

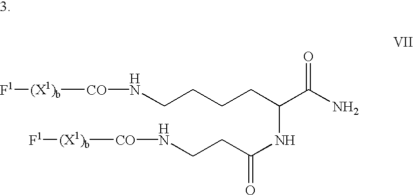

4. One or more peptidyl [—C(O)NR—] linkages (bonds) is replaced by a non-peptidyl linkage. Exemplary non-peptidyl linkages are —CH₂-carbamate [—CH₂—OC(O)NR—], phosphonate, —CH₂-sulfonamide [—CH₂—S(O)₂NR—], urea [—NHC(O)NH—], —CH₂-secondary amine, and alkylated peptide [—C(O)NR⁶— wherein R⁶ is lower alkyl].

5. The N-terminus is derivatized. Typically, the N-terminus may be acylated or modified to a substituted amine. Exemplary N-terminal derivative groups include —NRR¹ (other than —NH₂), —NRC(O)R¹, —NRC(O)OR¹, —NRS(O)₂R¹, —NHC(O)NHR¹, succinimide, or benzyloxycarbonyl-NH— (CBZ—NH—), wherein R and R¹ are each independently hydrogen or lower alkyl and wherein the phenyl ring may be substituted with 1 to 3 substituents selected from the group consisting of C₁-C₄ alkyl, C₁-C₄ alkoxy, chloro, and bromo.

6. The free C-terminus is derivatized. Typically, the C-terminus is esterified or amidated. For example, one may use methods described in the art to add (NH—CH₂—CH₂—NH₂)₂ to compounds of this invention having any of SEQ ID NOS: 504 to 508 at the C-terminus. Likewise, one may use methods described in the art to add —NH₂ to compounds of this invention having any of SEQ ID NOS: 924 to 955, 963 to 972, 1005 to 1013, or 1018 to 1023 at the C-terminus. Exemplary C-terminal derivative groups include, for example, —C(O)R² wherein R² is lower alkoxy or —NR³R⁴ wherein R³ and R⁴ are independently hydrogen or C₁-C₈ alkyl (preferably C₁-C₄ alkyl).

7. A disulfide bond is replaced with another, preferably more stable, cross-linking moiety (e.g., an alkylene). See, e.g., Bhatnagar et al. (1996), J. Med. Chem. 39: 3814-9; Alberts et al. (1993) Thirteenth Am. Pep. Symp., 357-9.

8. One or more individual amino acid residues is modified. Various derivatizing agents are known to react specifically with selected sidechains or terminal residues, as described in detail below.

Lysinyl residues and amino terminal residues may be reacted with succinic or other carboxylic acid anhydrides, which reverse the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with any one or combination of several conventional reagents, including phenylglyoxal, 2,3-butanedione, 1,2-cyclohexanedione, and ninhydrin. Derivatization of arginyl residues requires that the reaction be performed in alkaline conditions because of the high pKa of the guanidine functional group. Furthermore, these reagents may react with the groups of lysine as well as the arginine epsilon-amino group.

Specific modification of tyrosyl residues has been studied extensively, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl sidechain groups (aspartyl or glutamyl) may be selectively modified by reaction with carbodiimides (R′—N═C═N—R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Cysteinyl residues can be replaced by amino acid residues or other moieties either to eliminate disulfide bonding or, conversely, to stabilize cross-linking. See, e.g., Bhatnagar et al. (1996), J. Med. Chem. 39: 3814-9.

Derivatization with bifunctional agents is useful for cross-linking the peptides or their functional derivatives to a water-insoluble support matrix or to other macromolecular vehicles. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), pp. 79-86 (1983).

Compounds of the present invention may be changed at the DNA level, as well. The DNA sequence of any portion of the compound may be changed to codons more compatible with the chosen host cell. For E. coli, which is the preferred host cell, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Methods of Making

The compounds of this invention largely may be made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the peptides in an appropriate host. The vector comprises the DNA molecule that codes for the peptides operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides.

Compounds that contain derivatized peptides or which contain non-peptide groups may be synthesized by well-known organic chemistry techniques.

Uses of the Compounds

The compounds of this invention will have uses as described for laminin, echistatin, integrin antagonists, cell adhesion antagonists, and selectin antagonists known in the art. In particular, compounds of this invention are useful in treating:

conditions beneficially treated by inhibition of aggregation, including inhibition of platelet aggregation;

conditions beneficially treated by inhibition of angiogenesis (e.g., tumor growth, tumor metastasis);

inflammatory and autoimmune conditions (e.g., rheumatoid arthritis);

various forms of osteoporosis, such as:

primary osteoporosis;

post-menopausal and age-related osteoporosis;

endocrine osteoporosis (hyperthyroidism, hyperparathyroidism, Cushing's syndrome, and acromegaly);

hereditary and congenital forms of osteoporosis (e.g., osteogenesis imperfecta, homocystinuria, Menkes' syndrome, and Riley-Day syndrome);

osteoporosis due to immobilization of extremities;

osteoporosis secondary to other disorders, such as hemochromatosis, hyperprolactinemia, anorexia nervosa, thyrotoxicosis, diabetes mellitus, celiac disease, inflammatory bowel disease, primary biliary cirrhosis, rheumatoid arthritis, ankylosing spondylitis, multiple myeloma, lymphoproliferative diseases, and systemic mastocytosis;

osteoporosis secondary to surgery (e.g., gastrectomy) or to drug therapy, such as chemotherapy, anticonvulsant therapy, immunosuppressive therapy, and anticoagulant therapy; and the like.

In addition to therapeutic uses, the compounds of the present invention are useful in diagnosing diseases characterized by dysfunction of their associated protein of interest. In one embodiment, a method of detecting in a biological sample a protein of interest (e.g., a receptor) that is capable of being activated comprising the steps of: (a) contacting the sample with a compound of this invention; and (b) detecting activation of the protein of interest by the compound. The biological samples include tissue specimens, intact cells, or extracts thereof. The compounds of this invention may be used as part of a diagnostic kit to detect the presence of their associated proteins of interest in a biological sample. Such kits employ the compounds of the invention having an attached label to allow for detection.

### Pharmaceutical Compositions

In General. The present invention also provides methods of using pharmaceutical compositions of the inventive compounds. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations.

Oral dosage forms. Contemplated for use herein are oral solid dosage forms, which are described generally in Chapter 89 of Remington's Pharmaceutical Sciences (1990), 18th Ed., Mack Publishing Co. Easton Pa. 18042, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given in Chapter 10 of Marshall, K., Modern Pharmaceutics (1979), edited by G. S. Banker and C. T. Rhodes, herein incorporated by reference. In general, the formulation will include the inventive compound, and inert ingredients which allow for protection against the stomach environment, and release of the biologically active material in the intestine.

Also specifically contemplated are oral dosage forms of the above inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, N.Y., pp 367-83; Newmark, et al. (1982), J. Appl. Biochem. 4:185-9. Other polymers that could be used are poly-1,3-dioxolane and poly-1,3,6-tioxocane. Preferred for pharmaceutical usage, as indicated above, are PEG moieties.

For oral delivery dosage forms, it is also possible to use a salt of a modified aliphatic amino acid, such as sodium N-(8-[2-hydroxybenzoyl]amino) caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The compounds of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the compound of the invention with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the compound of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives may also be included in the formulation to enhance uptake of the compound. Additives potentially having this property are for instance the fatty acids oleic acid, linoleic acid and linolenic acid.

Controlled release formulation may be desirable. The compound of this invention could be incorporated into an inert matrix which permits release by either diffusion or leaching mechanisms e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Pulmonary delivery forms. Also contemplated herein is pulmonary delivery of the present protein (or derivatives thereof). The protein (or derivative) is delivered to the lungs of a mammal while inhaling and traverses across the lung epithelial lining to the bloodstream. (Other reports of pulmonary delivery include Adjei et al., Pharma. Res. (1990) 7: 565-9; Adjei et al. (1990), Internatl. I. Pharmaceutics 63: 135-44 (leuprolide acetate); Braquet et al. (1989), J. Cardiovasc. Pharmacol. 13 (suppl.5): s.143-146 (endothelin-1); Hubbard et al. (1989), Annals Int. Med. 3: 206-12 (α1-antitrypsin); Smith et al. (1989), J. Clin. Invest. 84: 1145-6 (α1-proteinase); Oswein et al. (March 1990), “Aerosolization of Proteins”, Proc. Symp. Resp. Drug Delivery II, Keystone, Colo. (recombinant human growth hormone); Debs et al. (1988), J. Immunol. 140: 3482-8 (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably 0.5 to 5 μm, for most effective delivery to the distal lung.

Pharmaceutically acceptable carriers include carbohydrates such as trehalose, mannitol, xylitol, sucrose, lactose, and sorbitol. Other 5 ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., 50 to 90% by weight of the formulation.

Nasal delivery forms. Nasal delivery of the inventive compound is also contemplated. Nasal delivery allows the passage of the protein to the blood stream directly after administering the therapeutic product to the nose, without the necessity for deposition of the product in the lung. Formulations for nasal delivery include those with dextran or cyclodextran. Delivery via transport across other mucous membranes is also contemplated.

Buccal delivery forms. Buccal delivery of the inventive compound is also contemplated. Buccal delivery formulations are known in the art for various peptides.

Dosages. The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

Specific preferred embodiments

The inventors contemplate preferred molecules having different peptide sequences attached to a vehicle. For example, a preferred molecule may include the sequences

| | |
|---|---|
| F¹-A-YIGSR-A-RGD | (SEQ ID NO:95) |
| YIGSR-A-RGD-A-F¹ | (SEQ ID NO:96) |

wherein “F¹” is an Fc domain as described previously herein and “Λ” is a linker as described previously herein.

All of the compounds of this invention can be prepared by methods described in PCT appl. no. WO 99/25044, filed Oct. 22, 1999, which is incorporated by reference in its entirety.

The invention will now be further described by the following working examples, which are illustrative rather than limiting. All of the information in the following working examples, including the processes and assays, may be applied to other compounds within this invention.

### EXAMPLE 1

### Preparation of Echistatin Fc-peptide Constructs

A synthetic gene encoding echistatin was fused via a 5 glycine linker to the C-terminus of the Fc portion of the human IgG1 molecule by PCR. The following oligonucleotides were used to form the echistatin template for a two-stage PCR reaction (Jayaraman K, Puccini C J., Biotechniques March 1992; 12(3):392-398. A PCR-mediated gene synthesis strategy involving the assembly of oligonucleotides representing only one of the strands.)

| | | |
|---|---|---|
| 2304-46 | GGG GGG CAT ATG GAA TGT GAA TCT GGT CCA TGC TGC | (SEQ ID NO: 97) |
| | AGA AAC TG | |
| 2304-47 | TAA GTT CTT GAA GGA AGG TAC CAT CTG TAA GAG AGC | (SEQ ID NO: 98) |
| | TAG AGG TG | |
| 2304-48 | ACG ACA TGG ACG ACT ACT GTA ACG GTA AGA CCT GTG | (SEQ ID NO: 99) |
| | ACT GCC CG | |
| 2304-49 | AGA AAC CCA CAC AAG GGT CCA GCT ACT TAA TOG ATC | (SEQ ID NO: 100) |
| | CGC GGC CGC CCA GCT | |

The single stranded template was assembled using the bridging oligonucleotides shown below:

| | | |
|---|---|---|
| 2304-52 | TTC AAG AAC TTA CAG TTT CTG CAG | (SEQ ID NO: 101) |
| 2304-53 | CGT CCA TGT CGT CAC CTC TAG CTC | (SEQ ID NO: 102) |
| 2304-54 | GTG TGG GTT TCT CGG GCA GTC ACA | (SEQ ID NO: 103) |

This template mixture was subjected to PCR using the following oligonucleotide primers:

| | | |
|---|---|---|
| 2305-26 | CCG GGT AAA GGT GGA GGT GGT GGT | (sense; SEQ ID NO: 104) |
| | GAA TGT GAA TCT GGT CCA TGC TGC | |
| 2304-51 | AGC TGG GCG GCC GCG GAT CCA | (antisense; SEQ ID NO: 105) |

The Fc portion of the construct was obtained via PCR using Amgen Strain #3728 (see WO 00/24770, published May 4, 2000 Patent Application A-533) as the template and the oligonucleotide primers

| | | |
|---|---|---|
| 1216-52 | AAC ATA AGT ACC TGT AGG ATC G | (SEQ ID NO:106) |
| 2305-27 | GCA GCA TGG ACC AGA TTC ACA TTC ACC ACC ACC TCC | (SEQ ID NO:107) |
| | ACC TTT ACC CGG A | |

The oligonucleotides 2305-26 and 2305-27 are fully complementary, allowing the two genes to be fused together in the correct reading frame by combining the above PCR products in a third reaction using the outside primers 1216-52 and 2304-51. The final PCR gene product (the full length fusion gene) was digested with restriction endonucleases NdeI and BamHI, and then ligated into the vector pAMG21 (described below) and transformed into competent E. coli strain 2596 (GM221, described herein) by electroporation. Clones were screened for the ability to produce the recombinant protein product and to possess the gene fusion having the correct nucleotide sequence. A single such clone was selected and designated Amgen strain #4592.

The nucleotide (SEQ ID NO: 108) and amino acid (SEQ ID NO: 109) sequences of the resulting fusion protein are shown below.

| | | |
|---|---|---|
| | NdeI | |
| | \| | |
| | CATATCGACAAAACTCACACATGTCCACCTTGTCCACCTCCGGAACTCCTGGGGCGACCG | |
| 1 | -+--------+---------+---------+---------+---------+--------- | 60 |
| | GTATACCTGTTTTGAGTGTGTACAGGTGGAACAGGTCGAGCCCTTGAGGACCcCccTGGc | |
| | M D K T H T C P P C P A P E L L C G P- | |
| | TCAGTCTTCCTCTTCCCCCCAAAACCCAAGCACACCCTCATCATCTCCCGGACCCCTGAG | |
| 61 | -+--------+---------+---------+---------+---------+--------- | 120 |
| | AGTCAGAAGGAGAAGCGGGGTTTTGGGTTCCTGTGGGAGTACTAGAGGGCCTGGGGACTC | |
| | S V F L F P P K P K D T L M I S R T P E- | |
| | GTCACATGCGTGGTGGTGGACGTGAGCCACGAAGACCCTGAGGTCAAGTTCAACTGGTAC | |
| 121 | -+--------+---------+---------+---------+---------+--------- | 180 |
| | CAGTGTACGCACCACCACCTGCACTCGGTGCTTCTGGGACTCCAGTTCAAGTTGACCATG | |
| | VT CV V V DV SHED P E V K F N WY - | |
| | CTGGACGGCGTGGAGGTGCATAATGCCAAGACAAAGCCGCGGGAGGAGCAGTACAACAGC | |
| 181 | -+--------+---------+---------+---------+---------+--------- | 240 |
| | CACCTGCCGCACCTCCACCTATTACGGTTCTGTTTCGGCGCCCTCCTCCTCATGTTGTCG | |
| | V D G V E V H N A K T K P R E E Q Y N S- | |
| | ACGTACCGTGTGGTCAGCCTCCTCACCGTCCTGCACCAGCACTGGCTGAATGGCAAGCAG | |
| 241 | -+--------+---------+---------+---------+---------+--------- | 300 |
| | TGCATGGCACACCAGTCGCAGGACTGGCACGACGTGGTCCTGACCGACTTACCGTTCCTC | |
| | T Y R V V S V L T V L H Q D W L N G K E- | |
| | TACAAGTGCAAGGTCTCCAACAAAGCCCTCCCAGCCCCCATCGAGAAAACCATCTCCAAA | |
| 301 | -+--------+---------+---------+---------+---------+--------- | 360 |
| | ATGTTCACGTTCCAGAGGTTGTTTCGGGAGGGTCGGGGGTAGCTCTTTTGGTAGAGGTTT | |
| | Y K C K V S N K A L P A P I E K T I S K- | |
| | GCCAAAGGGCAGCCCCGAGAACCACAGGTGTACACCCTGCCCCCATCCCGGGATGAGCTG | |
| 361 | -+--------+---------+---------+---------+---------+--------- | 420 |
| | CGGTTTCCCGTCGGGGCTCTTGGTGTCCACATGTGGGACGGGGGTAGGGCCCTACTCGAC | |
| | A K G Q P R E P Q V Y T L P P S R D E L- | |
| | ACCAAGAACCAGGTCAGCCTGACCTGCCTGGTCAAAGGCTTCTATCCCAGCGACATCGCC | |
| 421 | -+--------+---------+---------+---------+---------+--------- | 480 |
| | TGGTTCTTGGTCCAGTCGGACTGGACGGACCAGTTTCCGAAGATAGGGTCGCTGTAGCGG | |
| | T K N Q V S L T C L V K G F Y P S D I A- | |
| | GTGGAGTGGGAGAGCAATGGGCAGCCGGAGAACAACTACAAGACCACGCCTCCCGTGCTG | |
| 481 | -+--------+---------+---------+---------+---------+--------- | 540 |
| | CACCTCACCCTCTCGTTACCCGTCGGCCTCTTGTTGATGTTCTGGTGCGGAGGGCACGAC | |
| | V E W E S N G Q P E N N Y K T T P P V L- | |
| | GACTCCGACGGCTCCTTCTTCCTCTACAGCAAGCTCACCGTGGACAAGAGCAGGTGGCAG | |
| 541 | -+--------+---------+---------+---------+---------+--------- | 600 |
| | CTGAGGCTGCCGAGGAAGAAGGAGATGTCGTTCGAGTGGCACCTGTTCTCGTCCACCGTC | |
| | D S D G S F F L Y S K L T V D K S R W Q- | |
| | CAGGGGAACGTCTTCTCATGCTCCGTGATGCATGAGGCTCTGCACAACCACTACACGCAG | |
| 601 | -+--------+---------+---------+---------+---------+--------- | 660 |
| | GTCCCCTTGCAGAAGAGTACGAGGCACTACGTACTCCGAGACGTGTTGGTGATGTGCGTC | |
| | Q G N V F S C S V M H E A L H N H Y T Q- | |
| | AAGAGCCTCTCCCTGTCTCCGGGTAAAGGTGGAGGTGGTGGTGAATGTGAATCTGGTCCA | |
| 661 | -+--------+---------+---------+---------+---------+--------- | 720 |
| | TTCTCGGAGAGGGACAGAGGCCCATTTCCACCTCCACCACCACTTACACTTAGACCAGGT | |
| | K S L S L S P G K G G G G G E C E S G P- | |
| | End of Fc------------\| \|--gly linker-\| \|-----echistatin--+22 | |
| | TGCTGCAGAAACTGTAAGTTCTTGAAGGAAGGTACCATCTGTAAGAGAGCTAGAGGTGAC | |
| 721 | -+--------+---------+---------+---------+---------+--------- | 780 |
| | ACGACGTCTTTGACATTCAAGAACTTCCTTCCATGGTAGACATTCTCTCGATCTCCACTG | |
| | C C R N C K F L K E G T I C K R A R G D- | |
| | GACATGGACGACTACTGTAACGGTAAGACCTGTGACTGCCCGAGAAACCCACACAAGGGT | |
| 781 | -+--------+---------+---------+---------+---------+--------- | 840 |
| | CTGTACCTGCTGATGACATTGCCATTCTGGACACTGACGGGCTCTTTGGGTGTGTTCCCA | |
| | D M D D Y C N G K T C D C P R N P H K G- | |
| | BamHI | |
| | \| | |
| | CCAGCTACTTAATGGATCC | |
| 841 | -+---------+------- | |
| | GGTCGATGAATTACCTAGG | |
| | P A T * | |

Expression in E. coli. Cultures of the Fc-echistatin fusion constructs in E. coli GM221 were grown at 37° C. in Luria Broth medium. Induction of gene product expression from the 1uxPR promoter was achieved following the addition of the synthetic autoinducer N-(3-oxohexanoyl)-DL-homoserine lactone to the culture media to a final concentration of 20 ng/ml. Cultures were incubated at 37° C. for a further 3 hours. After 3 hours, the bacterial cultures were examined by microscopy for the presence of inclusion bodies and were then collected by centrifugation. Refractile inclusion bodies were observed in induced cultures indicating that the fusion protein was most likely produced in the insoluble fraction in E. coli. Cell pellets were lysed directly by resuspension in Laemmli sample buffer containing 10% β-mercaptoethanol and were analyzed by SDS-PAGE. An intense coomassie-stained band of the appropriate molecular weight was observed on an SDS-PAGE gel.

pAMG21. The expression plasmid pAMG21 can be derived from the Amgen expression vector pCFM1656 (ATCC #69576) which in turn be derived from the Amgen expression vector system described in U.S. Pat. No. 4,710,473. The pCFM1656 plasmid can be derived from the described pCFM836 plasmid (U.S. Pat. No. 4,710,473) by:

(a) destroying the two endogenous NdeI restriction sites by end filling with T4 polymerase enzyme followed by blunt end ligation;

(b) replacing the DNA sequence between the unique AatII and ClaI restriction sites containing the synthetic P_{L} promoter with a similar fragment obtained from pCFM636 (U.S. Pat. No. 4,710,473) containing the PL promoter (see SEQ ID NO: 110 below); and

(c) substituting the small DNA sequence between the unique ClaI and KpnI restriction sites with the oligonucleotide having the sequence of SEQ ID NO: 111.

```
SEQ ID NO:110:
AatII
5′ CTAATTCCGCTCTCACCTACCAAACAATGCCCCCCTGCAAAAAATAAATTCATAT-
3′ TGCAGATTAAGGCGAGAGTGGATGGTTTGTTACGGGGGGACGTTTTTTATTTAAGTATA-
      -AAAAAACATACAGATAACCATCTGCGGTGATAAATTATCTCTGGCGGTGTTGACATAA-
      -TTTTTTGTATGTCTATTGGTAGACGCCACTATTTAATAGAGACCGCCACAACTGTATTT-
      -TACCACTGGCGGTGATACTGAGCACAT 3′
      -ATGGTGACCGCCACTATGACTCGTGTAGC 5′
                                                                  ClaI
SEQ ID NO:111:
5′ CGATTTGATTCTAGAAGGAGGAATAACATATGGTTAACGCGTTGGAATTCGGTAC 3′
3′ TAAACTAAGATCTTCCTCCTTATTGTATACCAATTGCGCAACCTTAAGC 5′
      ClaI KpnI
```

The expression plasmid pAMG21 can then be derived from pCFM1656 by making a series of site-directed base changes by PCR overlapping oligo mutagenesis and DNA sequence substitutions. Starting with the BgIII site (plasmid bp #180) immediately 5′ to the plasmid replication promoter P_{copB} and proceeding toward the plasmid replication genes, the base pair changes are as shown in Table 7 below.

TABLE 7
Base pair changes resulting in pAMG21

| pAMG21 bp # | bp in pCFM1656 | bp changed to in pAMG21 |
|---|---|---|
| # 204 | T/A | C/G |
| # 428 | A/T | G/C |
| # 509 | G/C | A/T |
| # 617 | — | insert two G/Cbp |
| # 679 | G/C | T/A |
| # 980 | T/A | C/G |
| # 994 | G/C | A/T |
| # 1004 | A/T | C/G |
| # 1007 | C/G | T/A |
| # 1028 | A/T | T/A |
| # 1047 | C/G | T/A |
| # 1178 | G/C | T/A |
| # 1466 | G/C | T/A |
| # 2028 | G/C | bp deletion |
| # 2187 | G/C | T/A |
| # 2480 | A/T | T/A |
| # 2499-2502 | AGTG | GTCA |
| | TCAC | CAGT |
| # 2642 | TCCGAGC | 7 bp deletion |
| | AGGCTCG | |
| # 3435 | G/C | A/T |
| # 3446 | G/C | A/T |
| # 3643 | A/T | T/A |

The DNA sequence between the unique AatII (position #4364 in pCFM1656) and SacII (position #4585 in pCFM1656) restriction sites is substituted with the DNA sequence (SEQ ID NO: 112) shown below.

```
[AatII sticky end] 5′ GCGTAACGTATGCATGGTCTCC
(position #4358 in pAMG21) 3′ TGCACGCATTGCATACGTACCAGAGG
-CCATGCGAGAGTAGGGAACTGCCAGGCATCAAATAAAACGAAAGGCTCAGTCGAAAGACT-
-GGTACGCTCTCATCCCTTGACGGTCCGTAGTTTATTTTGCTTTCCGAGTCAGCTTTCTGA-
-GGGCCTTTCGTTTTATCTGTTGTTTGTCGGTGAACGCTCTCCTGAGTAGGACAAATCCGC-
-CCCGGAAAGCAAAATAGACAACAAACAGCCACTTGCGAGAGGACTCATCCTGTTTAGGCG-
-CGGGAGCGGATTTGAACGTTGCGAAGCAACGGCCCGGAGGGTGGCGGGCAGGACGCCCGC-
-GCCCTCGCCTAAACTTGCAACGCTTCGTTGCCGGGCCTCCCACCGCCCGTCCTGCGGGCG-
-CATAAACTGCCAGGCATCAAATTAAGCAGAAGGCCATCCTGACGGATGGCCTTTTTGCGT-
-GTATTTGACGGTCCGTAGTTTAATTCGTCTTCCGGTAGGACTGCCTACCGGAAAAACGCA-
                                                                                            AatII
-TTCTACAAACTCTTTTGTTTATTTTTCTAAATACATTCAAATATGGACGTCGTACTTAAC-
-AAGATGTTTGAGAAAACAAATAAAAAGATTTATGTAAGTTTATACCTGCAGCATGAATTG-
-TTTTAAAGTATGGGCAATCAATTGCTCCTGTTAAAATTGCTTTAGAAATACTTTGGCAGC-
-AAAATTTCATACCCGTTAGTTAACGAGGACAATTTTAACGAAATCTTTATGAAACCGTCG-
-GGTTTGTTGTATTGAGTTTCATTTGCGCATTGGTTAAATGGAAAGTGACCGTGCGCTTAC-
-CCAAACAACATAACTCAAAGTAAACGCGTAACCAATTTACCTTTCACTGGCACGCGAATG-
-TACAGCCTAATATTTTTGAAATATCCCAAGAGCTTTTTCCTTCGCATGCCCACGCTAAAC-
-ATGTCGGATTATAAAAACTTTATAGGGTTCTCGAAAAAGGAAGCGTACGGGTGCGATTTG-
-ATTCTTTTTCTCTTTTGGTTAAATCGTTGTTTGATTTATTATTTGCTATATTTATTTTTC-
-TAAGAAAAAGAGAAAACCAATTTAGCAACAAACTAAATAATAAACGATATAAATAAAAAG-
-GATAATTATCAACTAGAGAAGGAACAATTAATGGTATGTTCATACACGCATGTAAAAATA-
-CTATTAATAGTTGATCTCTTCCTTGTTAATTACCATACAAGTATGTGCGTACATTTTTAT-
-AACTATCTATATAGTTGTCTTTCTCTGAATGTGCAAAACTAAGCATTCCGAAGCCATTAT-
-TTGATAGATATATCAACAGAAAGAGACTTACACGTTTTGATTCGTAAGGCTTCGGTAATA-
-TAGCAGTATGAATAGGGAAACTAAACCCAGTGATAAGACCTGATGATTTCGCTTCTTTAA-
-ATCGTCATACTTATCCCTTTGATTTGGGTCACTATTCTGGACTACTAAAGCGAAGAAATT-
-TTACATTTGGAGATTTTTTATTTACAGCATTGTTTTCAAATATATTCCAATTAATCGGTG-
-AATGTAAACCTCTAAAAAATAAATGTCGTAACAAAAGTTTATATAAGGTTAATTAGCCAC-
-AATGATTGGAGTTAGAATAATCTACTATAGGATCATATTTTATTAAATTAGCGTCATCAT-
-TTACTAACCTCAATCTTATTAGATGATATCCTAGTATAAAATAATTTAATCGCAGTAGTA-
-AATATTGCCTCCATTTTTTAGGGTAATTATCCAGAATTGAAATATCAGATTTAACCATAG-
-TTATAACGGAGGTAAAAAATCCCATTAATAGGTCTTAACTTTATAGTCTAAATTGGTATC-
-AATGAGGATAAATGATCGCGAGTAAATAATATTCACAATGTACCATTTTAGTCATATCAG-
-TTACTCCTATTTACTAGCGCTCATTTATTATAAGTGTTACATGGTAAAATCAGTATAGTC-
-ATAAGCATTGATTAATATCATTATTGCTTCTACAGGCTTTAATTTTATTAATTATTCTGT-
-TATTCGTAACTAATTATAGTAATAACGAAGATGTCCGAAATTAAAATAATTAATAAGACA-
-AAGTGTCGTCGGCATTTATGTCTTTCATACCCATCTCTTTATCCTTACCTATTGTTTGTC-
-TTCACAGCAGCCGTAAATACAGAAAGTATGGGTAGAGAAATAGGAATGGATAACAAACAG-
-GCAAGTTTTGCGTGTTATATATCATTAAAACGGTAATAGATTGACATTTGATTCTAATAA-
-CGTTCAAAACGCACAATATATAGTAATTTTGCCATTATCTAACTGTAAACTAAGATTATT-
-ATTGGATTTTTGTCACACTATTATATCGCTTGAAATACAATTGTTTAACATAAGTACCTG-
-TAACCTAAAAACAGTGTGATAATATAGCGAACTTTATGTTAACAAATTGTATTCATGGAC-
-TAGGATCGTACAGCTTTACGCAAGAAAATGGTTTGTTATAGTCGATTAATCGATTTGATT-
-ATCCTAGCATGTCCAAATGCGTTCTTTTACCAAACAATATCAGCTAATTAGCTAAACTAA-
-CTAGATTTGTTTTAACTAATTAAAGGAGGAATAACATATGGTTAACGCGTTGGAATTCGA-
-GATCTAAACAAAATTGATTAATTTCCTCCTTATTGTATACCAATTGCGCAACCTTAAGCT-
                                                                                                  SacII
-GCTCACTAGTGTCGACCTGCAGGGTACCATGGAAGCTTACTCGAGGATCCGCGGAAAGAA-
-CGAGTGATCACAGCTGGACGTCCCATGGTACCTTCGAATGAGCTCCTAGGCGCCTTTCTT-
-GAAGAAGAAGAAGAAAGCCCGAAAGGAAGCTGAGTTGGCTGCTGCCACCGCTGAGCAATA-
-CTTCTTCTTCTTCTTTCGGGCTTTCCTTCGACTCAACCGACGACGGTGGCGACTCGTTAT-
-ACTAGCATAACCCCTTGGGGCCTCTAAACGGGTCTTGAGGGGTTTTTTGCTGAAAGGAGG-
-TGATCGTATTGGGGAACCCCGGAGATTTGCCCAGAACTCCCCAAAAAACGACTTTCCTCC-
-AACCGCTCTTCACGCTCTTCACGC 3′ [SacII sticky end]
-TTGGCGAGAAGTGCGAGAAGTG 5′ (position #5904 in pAMG21)
```

During the ligation of the sticky ends of this substitution DNA sequence, the outside AatII and SacII sites are destroyed. There are unique AatII and SacII sites in the substituted DNA.

GM221 (Amgen #2596). The Amgen host strain #2596 is an E. coli K-12 strain derived from Amgen strain #393. It has been modified to contain both the temperature sensitive lambda repressor cI857s7 in the early ebg region and the lacI^{Q} repressor in the late ebg region (68 minutes). The presence of these two repressor genes allows the use of this host with a variety of expression systems, however both of these repressors are irrelevant to the expression from 1uxP_{R}. The untransformed host has no antibiotic resistances.

The ribosome binding site of the cI857s7 gene has been modified to include an enhanced RBS. It has been inserted into the ebg operon between nucleotide position 1170 and 1411 as numbered in Genbank accession number M64441Gb_Ba with deletion of the intervening ebg sequence. The sequence of the insert is shown below with lower case letters representing the ebg sequences flanking the insert shown below

| | |
|---|---|
| ttattttcgtGCGGCCGCACCATTATCACCGCCAGAGGTAAACTAGTCAACACGCACGGTGTTAGATATTTAT | (SEQ ID NO:113): |
| CCCTTGCGGTGATAGATTGACCACATCCATTTGATTCTAGAAGGAGGGATAATATATGAGCACAAAAAAGAAA | |
| CCATTAACACAAGAGCAGCTTGAGGACGCACGTCGCCTTAAAGCAATTTATGAAAAAAAGAAAAATGAACTTG | |
| GCTTATCCCAGGAATCTGTCGCAGACAAGATGGGGATGGGGCACTCAGGCGTTGGTGCTTTATTTAATGGCAT | |
| CAATGCATTAAATGCTTATAACGCCGCATTGCTTACAAAAATTCTCAAAGTTAGCGTTGAAGAATTTAGCCCT | |
| TCAATCGCCAGAGAATCTACGAGATGTATGAAGCGGTTAGTATGCAGCCGTCACTTAGAAGTGAGTATGAGTA | |
| CCCTGTTTTTTCTCATGTTCACGCAGGGATGTTCTCACCTAAGCTTAGAACCTTTACCAAAGGTGATGCGGAG | |
| AGATGGGTAAGCACAACCAAAAAAGCCAGTGATTCTGCATTCTCGCTTGAGGTTGAAGGTAATTCCATGACCG | |
| CACCAACAGGCTCCAAGCCAAGCTTTCCTCACGGAATGTTAATTCTCGTTGACCCTGAGCAGGCTGTTGAGCC | |
| AGGTGATTTCTGCATAGCCAGACTTCGGGCTCATGAGTTTACCTTCAAGAAACTGATCAGGGATAGCGGTCAG | |
| GTGTTTTTACAACCACTAAACCCACAGTACCCAATGATCCCATGCAATGAGAGTTGTTCCGTTGTGGGGAAAG | |
| TTATCGCTAGTCAGTGGCCTGAAGAGACGTTTGGCTGATAGACTAGTGGATCCACTAGTgtttctgccc | |

The construct was delivered to the chromosome using a recombinant phage called MMebg-cI857s7enhanced RBS #4 into F′tet/393. After recombination and resolution only the chromosomal insert described above remains in the cell. It was renamed F′tet/GM101. F′tet/GM101 was then modified by the delivery of a lacI^{Q} construct into the ebg operon between nucleotide position 2493 and 2937 as numbered in the Genbank accession number M64441Gb_Ba with the deletion of the intervening ebg sequence. The sequence of the insert is shown below with the lower case letters representing the ebg sequences flanking the insert (SEQ ID NO: 114) shown below:

ggcggaaaccGACGTCCATCCAATGCTGCAAAACCTTTCGCGGTATGGCATGATAGCGCCCGCAAGAGAGTCA
ATTCAGGGTGGTGAATGTCAAACCAGTAACGTTATACGATGTCGCAGACTATGCCCGTGTCTCTTATCAGACC
GTTTCCCGCGTGGTGAACCAGGCCAGCCACGTTTCTGCGAAAACGCGGGAAAAAGTCGAAGCGGCGATGGCGG
AGCTGAATTACATTCCCAACCGCGTGGCACAACAACTGGCGGGCAAACAGTCGCTCCTGATTGGCGTTGCCAC
CTCCAGTCTGGCCCTGCACGCGCCGTCGCAAATTGTCGCGGCGATTAAATCTCGCGCCGATCAACTGGGTGCC
AGCGTGGTGGTGTCGATGGTAGAACGAAGCGGCGTCGAAGCCTGTAAAGCGGCGGTGCACAATCTTCTCGCGC
AACGCGTCAGTGGGCTGATCATTAACTATCCGCTGGATGACCAGGATGCCATTGCTGTGGAAGCTGCCTGCAC
TAATGTTCCGGCGTTATTTCTTGATGTCTCTGACCAGACACCCATCAACAGTATTATTTTCTCCCATGAAGAC
GGTACGCGACTGCGCGTGGAGCATCTGGTCGCATTGGGTCACCAGCAAATCGCGCTGTTAGCGGGCCCATTAA
GTTCTGTCTCGGCGCGTCTGCGTCTCGCTGGCTCCCATAAATATCTCACTCGCAATCAAATTCAGCCGATAGC
GGAACGGGAAGGCGACTGGAGTGCCATGTCCGGTTTTCAACAAACCATGCAAATGCTGAATGAGGGCATCGTT
CCCACTGCOATGCTCGTTGCCAACCATCAGATGGCGCTGGGCGCAATGCGCCCCATTACCGAGTCCGCGCTGC
GCGTTGGTGCGGATATCTCGGTAGTGGGATACGACGATACCGAAGACAGCTCATGTTATATCCCGCCGTTAAC
CACCATCAAACAGCATTTTCGCCTGCTGGGGCAAACCAGCGTGGACCCCTTGCTGCAACTCTCTCAGGGCCAG
GCGGTGAAGGGCAATCAGCTGTTCCCCGTCTCACTGGTGAAAAGAAAAACCACCCTGGCGCCCAATACGCAAA
CCGCCTCTCCCCGCGCGTTGGCCGATTCATTAATGCAGCTGGCACGACAGGTTTCCCGACTGGAAAGCGGACA
GTAAGGTACCATAGGATCCaggcaCagga

The construct was delivered to the chromosome using a recombinant phage called AGebg-LacIQ#5 into F′tet/GM101. After recombination and resolution only the chromosomal insert described above remains in the cell. It was renamed F′tet/GM221. The F′tet episome was cured from the strain using acridine orange at a concentration of 25 μg/ml in LB. The cured strain was identified as tetracyline sensitive and was stored as GM221.

Purification of Vitronectin. Vitronectin was prepared from out-dated human plasma as described by Yatohgo et al (1988) Struct. Funct. 13: 281-92, with modifications. Normal human blood collected in citrate tubes was centrifuged and clotted overnight with the addition of CaCl₂. The clot was centrifuged, filtered at 0.45 μm, and applied to a Heparin Sepharose column that was equilibrated with 10 mM NaPO₄, 5 mM EDTA, 0.13 M NaCl pH 7.7. The column flow through was collected as a single pool, urea was added to a final concentration of 8 M, and mixed overnight. The sample was then incubated with Heparin Sepharose which had been equilibrated with 10 mM NaPO₄, 5 mM EDTA, 8 M Urea pH 7.7 (buffer A) overnight. The Heparin Sepharose was separated from the liquid by centrifugation and washed once with buffer A, buffer A+0.13 M NaCl, and buffer A+0.13 NaCl and 10 mM BME. The vitronectin was eluted from the column with buffer A+0.5 M NaCl. The fractions containing Vitronectin were buffer exchanged into PBS and stored at −70° C.

Ruthenylation of Vitronectin and Fibrinogen. Purified human vitronectin or purified human fibrinogen (Calbiochem) was dialyzed into 50 mM borate, 100 mM NaCl pH 8.0. A stock solution of ruthenium (II) tris bipyridine N-hydroxysuccinimide ester (Origen TAG® Ester, Igen Inc. Gaithersburg, Md.) was freshly prepared by adding 50 μL DMSO to 150 μg of the Origen TAG-NHS ester. Fifty microliters of the Origen TAG-NHS ester was added to one fifth molar ratio of the matrix protein. After one hour incubation at 250° C., the reaction was quench by the addition of 50 μL of 2 M glycine. Unincorporated ruthenium and excess glycine were removed by dialysis into PBS, 0.05% NaN₃. Protein concentrations were determined using Micro-BCA (Pierce, Rockford, Ill.). Origen TAG incorporation was assessed at 455 nm (e=13,700 M⁻¹ cm⁻¹). Vitronectin-Ru and Fibrinogen-Ru were stored at −70° C. until needed.

Purification of Platelet Fibrinogen Receptor αIIbβ3. Twelve units of outdated platelets were washed with phosphate-buffered saline (PBS) and centrifuged at low speed to remove red blood cells (RBCs). The washed platelets were lysed in, 20 mM Tris-HCl pH 7.4, 140 mM NaCl, 2 mM CaCl₂, 1 mM pefabloc, 3% octylglucoside with gentle stirring for two hours at 4° C. The lysate was centrifuged at 100,000×g for 1 hour to pellet insoluble cellular debris. The resulting supernatant was applied to a lentil lectin (EY labs) column and washed with lysis buffer containing 1% octylglucoside (binding buffer) until a stable UV baseline was reached. Purified αIIbβ3 was eluted from the column with binding buffer containing 10% dextrose. Purified αIIbβ3 was stored at −70° C. until needed.

Purification of αvβ3 and αvβ5. Frozen placentas were thawed overnight at 4° C., cut into 1 cm sections, and washed with 50 mM Tris-HCl, 100 mM NaCl, 1 mM PMSF pH 7.5 (buffer A). The placentas were then incubated overnight in buffer A with the addition of 3% (w/v) octyglucoside. Extracted protein was separated from whole tissue by centrifugation. The extract was then 0.45 μm filtered and NaN₃ was added to a final concentration of 0.02%. The sample was then loaded on to an anti-αvβ3 or anti-αvβ5 affinity column, washed with buffer A plus 1% (w/v) octyglucoside, and eluted with Gentle Elution Buffer® (Pierce). The fractions containing αvβ3 or αvβ5 were exchanged into buffer A plus 1% octylglucoside and stored at −70° C. Purified αvβ3 and αvβ5 were also purchased from Chemicon International Inc.

Incorporation of αvβ3, αvβ5, or αIIbβ3 on paramagnetic beads. The αvβ3, αvβ5, or αIIbβ3 paramagnetic beads were prepared from 4.5μ uncoated Dynabeads® (Dynal® Lake Success, N.Y.). Uncoated Dynabeads® were washed three times in phosphate buffered saline pH 7.4 (PBS) and resuspended in 50 mM Tris-HCl, 100 mM NaCl, 1 mM MgCl₂, 1 mM CaCl₂, and 1 mM MnCl₂ pH 7.5 (Buffer A). Purified receptor αvβ3, αvβ5(Chemicon), or αIIbβ3 were quickly diluted in buffer A and added to the uncoated Dynabeads® at a ratio of 50 μg protein to 10⁷ beads. The bead suspension was incubated with agitation overnight at 4° C. The beads were washed three times in buffer A, 0.1% bovine serum albumin (BSA) and resuspended buffer A+3% BSA. After three hours at 4° C. the beads were wash three times in Buffer A, 1% BSA, 0.05% azide and stored at −70° C. until needed.

Solid Phase Binding Assay. All compounds were dissolved and serially diluted in 100% DMSO prior to a final dilution in assay buffer (50 mM Tris-HCl pH 7.5, 100 mM NaCl, 1 mM CaCl₂, 1 mM Mg Cl₂, 1 mM MnCl₂, 1% BSA, 0.05% Tween-20) containing Vitronectin-Ru or Fibrinogen-Ru and appropriate integrin coated paramagnetic beads. The assay mixture was incubated at 25° C. for two hours with agitation and subsequently read on an Origen Analyzer® (Igen Inc. Gaithersburg, Md.) Non-specific binding was determined using 1 μM Vitronectin, 1 μM Fibrinogen or 5 mM EDTA. The data was prepared using a four-parameter fit by the Levenburg Marquardt algorithm (XLfit® ID Business Solutions.) Ki values were calculated using the equation of Cheng and Prusoff (1973) Biochem. Pharmacology 22: 3099-3108.

### EXAMPLE 2

Preparation of laminin-Fc fusions. The following laminin-related peptides were fused to the N-terminus of the Fc portion of the human IgG1 molecule by PCR.

MYIGSRGGGGG (SEQ ID NO: 115)

MYIGSRYIGSRYIGSR (SEQ ID NO: 116)

MYIGSRYIGSRYIGSRYIGSRYIGSR (SEQ ID NO: 117)

MIPCNNKGAHSVGLMWWMLARGGGGG (SEQ ID NO: 118)

MYIGSRREDVEILDVPDSGRGGGGG (SEQ ID NO: 119)

MRGDRGDYIGSRRGDGGGGG (SEQ ID NO: 120)

For these fusions, an unrelated Fc-peptide fusion (THF gamma 2-Fc) was used as the PCR template (Amgen strain #4490, described in WO 00/24782, published May 4, 2000). The sense oligonucleotides given below were each used in a standard PCR reaction with the antisense oligonucleotide 1200-54 to yield an in-frame fusion of the desired peptide to Fc.

| | | |
|---|---|---|
| 2453-79 | GAA TAA CAT ATG TAC ATC GGT TCT CGT GGT GGA GGC | (SEQ ID NO:121) |
| | GGT GGG GAC AAA | |
| 2554-70 | GAA TAA CAT ATG TAC ATC GGT TCT CGT TAT ATT GGC | (SEQ ID NO:122) |
| | TCC CGC TAC ATT GGT AGC CGT GAC AAA ACT CAC ACA | |
| | TGT CCA CCT | |
| 2554-71 | GAA TAA CAT ATG TAC ATC GGT TCT CGT TAT ATT GGC | (SEQ ID NO:123) |
| | TCC CGC TAC ATT GGT AGC CGT TAT ATC GGC TCT CGC | |
| | TAT ATT GGT AGC CGC GAC AAA ACT CAC ACA TGT CCA | |
| | CCT | |
| 2719-06 | GAA TAA CAT ATG ATC CCG TGC AAC AAC AAA GGT GCT | (SEQ ID NO:124) |
| | CAC TCT GTT GGT CTG ATG TGG TGG ATG CTG GCT CGT | |
| | GGT GGA GGC GGT GGG GAC AAA | |
| 2719-07 | GAA TAA CAT ATG TAC ATC GGT TCT CGT CGT GAA GAC | (SEQ ID NO:125) |
| | GTT GAA ATC CTG GAC GTT CCG GAC TCT GGT CGT GGT | |
| | GGA GGC GGT GGG GAC AAA | |
| 2719-08 | GAA TAA CAT ATG CGT GGT GAC CGT GGT GAC TAC ATC | (SEQ ID NO:126) |
| | GGT TCT CGT CGT GGT GAC GGT GGA GGC GGT GGG GAC | |
| | AAA | |
| 1200-54 | GTT ATT GCT CAG CGG TGG CA | (SEQ ID NO:127) |

Each PCR gene product (full length fusion gene) was digested with restriction endonucleases NdeI and BamHI, and then ligated into the vector pAMG21 (described above) and transformed into competent E. coli strain 2596 (GM221, described herein) by electroporation. Clones were screened for the ability to produce the recombinant protein product and to possess the gene fusion having the correct nucleotide sequence. Expression and purification of each fusion protein was carried out as described above.

Laminin activity assay: Apoptosis of HT-1080 human fibrosarcoma cells. HT-1080 cells from a human fibrosarcoma are cultured in DMEM supplemented with 10% fetal bovine serum, 100 μg/ml streptomycin and 100 units/ml penicillin. The culture is started at a density of 5×10⁴ cells per plate. The Fc-peptides (Fc-YIGSR, Fc-(YIGSR)₂, YIGSR-Fc, or (YIGSR)₂-Fc)) at various concentrations are added to each plate, and after 16 hours the cells are harvested for evaluation of apoptosis with crystal violet solution at 560 nm absorbance. The DNA fragmentation analysis is also carried out to assess the degree of apoptosis in a 1.5% agarose gel and visualized by ethidium bromide staining.

### EXAMPLE 3

### Preparation of Additional Laminin Peptibodies

Two additional laminin peptides, (YIGSR)₃ and (YIGSR)₅ from Table 6 herein, were fused to human IgG1. Those fusion peptides were designated as laminin-3 ((YIGSR)₃-Fc) and laminin-5 ((YIGSR)₅-Fc). The purified peptide-Fc fusions were examined for their effect on the growth of HT1080 cells as described in Example 1. The synthetic peptide (YIGSR)₃ gave an IC100 of 2.9 μM, whereas the IC100 of (YIGSR)₃-Fc was 55 nM. A 50-fold enhancement was seen after it was fused to human IgG1.

Since some proteolysis was seen in laminin-5, the IC100 of laminin-5 could not be assessed accurately. All of the degradation occurred after the arginine residue (at the junction between the YIGSR repeats). In order to eliminate the degradation, several different peptides were designed and synthesized. Some of them showed the inhibition of HT1080 cell growth. Two of the best peptides were

REDVEILDVYIGSRPDSGR (SEQ ID NO: 136) and

YIGSRREDVEILDVPDSGR (SEQ ID NO: 137).

### EXAMPLE 4

### Evaluation of Plasma Clearance

Synthetic peptide and Fc-peptides are iodinated with ¹²⁵I by lodogen method. The inhibitory effect of the iodinated molecules on HT-1080 cells are indistinguishable from those non-iodinated molecules. C57BL/6 mice are injected intravenously and subcutaneously with the iodinated peptide/Fc-peptides and blood is collected at various time points. The blood radioactivity is measured with γ-counter and the pharmacokinetic profiles of the injected molecules are evaluated.

### EXAMPLE 5

### Experimental Pulmonary Metastasis Assay

Highly metastatic and invasive B16-BL6 melanoma cells are suspended in MEM medium containing 0.1% BSA (1.5×10⁶ cells/ml). C57BL/6 mice are intravenously inoculated with the cell solution (0.1 ml). Following tumor inoculation, several concentrations of the peptides and various Fc-peptides are injected intravenously. Mice are scarified two to three weeks after tumor inoculation and colonies on the lung surface are evaluated.

The invention now being fully described, it will be apparent to one of ordinary skill in the art that many changes and modifications can be made thereto, without departing from the spirit and scope of the invention as set forth herein.

What is claimed is:

1. A composition of matter comprising
a. an integrin/adhesion antagonist peptide; and
b. a vehicle.

2. A composition of the formula
(X¹)ₐ—F¹—(X²)_{b}
and multimers thereof, wherein:
F¹ is an Fc domain;
X¹ and X² are each independently selected from —(L¹)_{c}—P¹, —(L¹)_{c}—P¹—(L²)_{d}—P², —(L¹)_{c}—P¹—(L²)_{d}—P²—(L³)ₑ—P³, and —(L¹)_{c}—P¹—(L²)_{d}—P²—(L³)ₑ—P³—(L⁴)_{f}—P⁴
P¹, P², P³, and P⁴ are each independently sequences of integrin/adhesion antagonist peptides;
L¹, L², L³, and L⁴ are each independently linkers; and
a, b, c, d, e, and f are each independently 0 or 1, provided that at least one of a and b is 1.

3. The composition of matter of claim 1 of the formulae
X¹—F¹
or
F¹—X².

4. The composition of matter of claim 3 of the formula
F¹—(L¹)_{c}—P¹.

5. The composition of matter of claim 3 of the formula
F¹—(L¹)_{c}—P¹—(L²)_{d}—P².

6. The composition of matter of claim 2 wherein F¹ is an Fc domain.

7. The composition of matter of claim 2 wherein F¹ is an IgG Fc domain.

8. The composition of matter of claim 2 wherein F¹ is an IgG1 Fc domain.

9. The composition of matter of claim 2 wherein F¹ comprises the sequence of SEQ ID NO: 2.

10. The composition of matter of claim 2 wherein X¹ and X² comprise one or more sequences selected from SEQ ID NOS: 7 to 21.

11. The composition of matter of claim 2 wherein the composition of matter comprises one or more sequences selected from SEQ ID NOS: 22 to 94.

12. The composition of matter of claim 2 wherein the composition of matter comprises one or more sequences selected from SEQ ID NOS: 7and 9 to 16.

13. The composition of matter of claim 2 wherein the composition of matter comprises one or more sequences selected from Tables 3, 4, 5, and 6 (SEQ ID NOS: 22 to 94, 128 to 137).

14. A DNA encoding a composition of matter of any of claims 6 to 13.

15. An expression vector comprising the DNA of claim 14.

16. A host cell comprising the expression vector of claim 15.

17. The cell of claim 16, wherein the cell is an E. coli cell.

18. A process for preparing a pharmacologically active compound, which comprises
a) selecting at least one randomized integrin/adhesion antagonist peptide; and
b) preparing a pharmacologic agent comprising at least one Fc domain covalently linked to at least one amino acid sequence of the selected peptide or peptides.

19. The process of claim 18, wherein the peptide is selected in a process comprising one or more techniques selected from yeast-based screening, rational design, protein structural analysis, screening of a phage display library, an E. coli display library, a ribosomal library, or a chemical peptide library.

20. The process of claim 18, wherein the preparation of the pharmacologic agent is carried out by:
a) preparing a gene construct comprising a nucleic acid sequence encoding the selected peptide and a nucleic acid sequence encoding an Fc domain; and
b) expressing the gene construct.

21. The process of claim 18, wherein the gene construct is expressed in an E. coli cell.

22. The process of claim 18 wherein the Fc domain is an IgG Fc domain.

23. The process of claim 18, wherein the vehicle is an IgG1 Fc domain.

24. The process of claim 18, wherein the vehicle comprises the sequence of SEQ ID NO: 2.

25. A composition of matter comprising an amino acid sequence selected from SEQ ID NOS: 132 to 137.
